(19) United States
(12) Patent Application Publication
(10) Pub. No.: US 2014/0275010 A1
(43) Pub. Date: Sep. 18, 2014

(54) QUATERNARY SALTS

(71) Applicants: Guo Zhu Zheng, Lexington, MA (US); Yuan Wang, Andover, MA (US); Gregg F. Keany, Lexington, MA (US); Kenzo Arai, Cambridge, MA (US); Kazunobu Kira, Brookline, MA (US); Xiang Liu, Winchester, MA (US); Nick Gearhart, Durango, CO (US); Baudouin Gerard, Cambridge, MA (US)

(72) Inventors: Guo Zhu Zheng, Lexington, MA (US); Yuan Wang, Andover, MA (US); Gregg F. Keany, Lexington, MA (US); Kenzo Arai, Cambridge, MA (US); Kazunobu Kira, Brookline, MA (US); Xiang Liu, Winchester, MA (US); Nick Gearhart, Durango, CO (US); Baudouin Gerard, Cambridge, MA (US)

(21) Appl. No.: 14/204,078

(22) Filed: Mar. 11, 2014

Related U.S. Application Data
(60) Provisional application No. 61/777,237, filed on Mar. 12, 2013.

(51) Int. Cl. C07D 407/06; C07D 405/14; C07D 487/08; C07D 487/10

(52) U.S. Cl. CPC C07D 407/06; C07D 487/10; C07D 405/14; C07D 487/08
USPC 514/210.18; 544/374; 514/254.1; 544/147; 514/231.5; 549/271; 514/450; 540/575; 514/218; 548/953; 546/208; 514/326; 546/187; 514/316; 548/453; 514/414

(57) ABSTRACT

The present invention provides quaternary salts, such as those of Formula I:

[chemical structure]

as well as the use thereof in methods of treatment for cancer.

### BACKGROUND OF THE INVENTION

In eukaryote organisms, newly synthesized messenger RNAs typically have multiple introns which are excised to provide the mature mRNA. The spliceosome is a multisubunit complex that accomplishes this task. The spliceosome consists of five small nuclear RNAs (snRNAs; U1-6) in combination with a variety of proteins.

Mutations in the splicing factor 3B subunit 1 (SF3B1) of the spliceosome have been described in a number of cancers. (“SF3B1 mutations in myelodysplastic syndromes: clinical associations and prognostic implications”, Damm F. et al. Leukemia, 2011, 1-4; “Frequent pathway mutations of splicing machinery in myelodysplasia”, Yoshida K. et al, Nature, 2011, 478, 64-69; “Clinical significance of SF3B1 mutations in myelodysplastic syndromes and myelodysplastic/myeloproliferative neoplasms”, Malcovati L. et al., Blood, 2011, 118, 24, 6239-6246; “Mutations in the spliceosome machinery, a novel and ubiquitous pathway in leukemogenesis”, Makishima et al, Blood, 2012, 119, 3203-3210; “Somatic SF3B1 mutation in myelodysplasia with ring sideroblasts”, Pappaemannuil, E. et al, New England J. Med. 2011, DOI 10.1056/NEJMoa1103283. “Defects in the spliceosomal machinery: a new pathway of leukaemogenesis”, Maciejewski, J. P., Padgett, R. A., Br. J. Haematology, 2012, 1-9; “Mutations in the SF3B1 splicing factor in chronic lymphocytic leukemia: associations with progression and fludarabine-refractoriness”, Rossi et al, Blood, 2011, 118, 6904-6908; “Exosome sequencing identifies recurrent mutations of the splicing factor SF3B1 gene in chronic lymphocytic leukemia”, Quesada et al, Nature Genetics, 2011, 44, 47-52; “Spliceosomal gene mutations are frequent events in the diverse mutational spectrum of chronic myelomonocytic leukemia but largely absent in juvenile myelomonocytic leukemia”, Kar S. A. et al, Haematologia, 2012, DOI: 10.3324/haematol.2012.064048. “Whole genome analysis informs breast cancer response to aromatase inhibition”, Ellis et al, Nature, 2012, 486, 353-360.) These findings have suggested targeting the spliceosome, including SF3B1, as a cancer therapeutic strategy. (“Targeting the Spliceosome”, Raymond, B. Nature Chemical Biology 2007, 3, 533-535.)

In 2004, researchers from the Tsukuba Research Laboratories of Eisai Inc. and Bioresource Laboratories of Mercian Corporation reported the discovery of seven compounds isolated from the bacteria Streptomyces platensis (Sakai, Takashi; Sameshima, Tomohiro; Matsufuji, Motoko; Kawamura, Naoto; Dobashi, Kazuyuki; Mizui, Yoshiharu. Pladienolides, New Substances from Culture of Streptomyces platensis Mer-11107.1. Taxonomy, Fermentation, Isolation and Screening. The Journal of Antibiotics. 2004, Vol. 57, No. 3.) These seven compounds, which have been termed pladienolides, were discovered while screening for inhibitors of the vascular endothelial growth factor (VEGF) promoter. Of the seven pladienolides discovered in this study, six inhibited expression of a reporter gene controlled by human VEGF promoter. These six compounds also inhibited proliferation of U251 human glioma cells in vitro. The most potent of these six compounds, Pladienolide B, inhibited VEGF-promoted gene expression with an IC₅₀ of 1.8 nM, and inhibited glioma cell proliferation with an IC₅₀ of 3.5 nM. The structure of pladienolide B was elucidated in a subsequent publication. (Sakai, Takashi; Sameshima, Tomohiro; Matsufuji, Motoko; Kawamura, Naoto; Dobashi, Kazuyuki; Mizui, Yoshiharu. Pladienolides, New Substances from Culture of Streptomyces platensis Mer-11107. II. Physico-chemical Properties and Structure Elucidation. The Journal of Antibiotics. Vol. 57, No. 3. (2004).) Pladienolide B was subsequently shown to target the SF3b spliceosome to inhibit splicing and alter the pattern of gene expression. (Kotake et al., “Splicing factor SF3b as a target of the antitumor natural product pladienolide”. Nature Chemical Biology 2007, 3, 570-575).

Certain pladienolide B analogs have been previously disclosed: WO 2002/060890; WO 2004/011459; WO 2004/011661; WO 2004/050890; WO 2005/052152; WO 2006/009276; WO 2008/126918.

There remains a need for the discovery and development of new compounds which target the spliceosome as potential anti-cancer therapeutic agents. It is an object of the present disclosure to provide such compounds, their method of making, and their therapeutic use.

### SUMMARY OF THE INVENTION

Provided herein according to some embodiments is a quaternary salt of Formula I:

[chemical structure]

wherein:

Y is a quaternary nitrogen;

X⁻ is a pharmaceutically acceptable anion;

L¹ is alkylene or alkenylene; or L¹ and R¹² are joined together to form a branched alkylene or alkenylene;

L² is H or alkyl; or L² and R¹² are joined together to form an alkylene or alkenylene; or L² and L¹ are joined together to form a branched alkylene or alkenylene;

or L¹, L² and R¹² are joined together, with the two nitrogens to which they are attached, to form a Spiro heterocycle or a bridged heterocycle;

R¹² is alkyl, or R¹² is joined to L¹ and/or L²;

R¹⁹ and R¹¹ are each independently: alkyl, alkenyl, cycloalkyl, aryl, arylalkyl, arylalkenyl, and heterocycle; or R¹⁰ and R¹¹ are taken together with the nitrogen to which they are attached to form a heterocycle;

R¹ is hydrogen, hydroxy, alkoxy, C₁₋₆ alkyl ester or alkyl;

R² is H or alkyl;

R³ is H, alkyl, or hydroxy;

R⁴, R⁵ and R⁸ are each independently alkyl (e.g., methyl, ethyl, propyl);

R⁶ and R⁷ are each independently selected from the group consisting of H, hydroxy, and alkyl (e.g., methyl, ethyl, propyl); and

R⁹ is hydroxy or oxo;

or a pharmaceutically acceptable prodrug thereof,

wherein said alkylene, alkenylene, alkyl, alkenyl, cycloalkyl, aryl, arylalkyl, arylalkenyl, and heterocycle groups may each independently be unsubstituted or substituted (e.g., 1-3 times).

In some embodiments, the salt is a salt of Formula I(a):

[chemical structure]

wherein:

X⁻ is a pharmaceutically acceptable anion;

R¹, R², R³, R⁴, R⁵, R⁶, R⁷, R⁸, and R⁹ are as defined above;

L¹ and L² are each independently C₁-C₆ alkylene or C₂-C₆ alkenylene; or L¹ and L² are taken together with the two nitrogens to which they are attached to form a bridged heterocyclic moiety;

R¹⁰ and R¹¹ are each independently selected from the group consisting of: C₁-C₆ alkyl, C₂-C₆ alkenyl, C₃-C₈ cycloalkyl, and C₃-C₈ heterocycle; or R₁₀ and R₁₁ are taken together with the nitrogen to which they are attached to form a 5-7 membered heterocycle;

or a pharmaceutically acceptable prodrug thereof.

In some embodiments, the salt is a salt of Formula I(b):

[chemical structure]

wherein:

X⁻ is a pharmaceutically acceptable anion;

R¹, R², R³, R⁴, R⁵, R⁶, R⁷, R⁸, and R⁹ are as defined above;

L¹ is C₁-C₆ alkylene or C₂-C₆ alkenylene;

R¹³ is selected from the group consisting of: hydrogen, C₁-C₆ alkyl, C₂-C₆ alkenyl, C₃-C₈ cycloalkyl, and C₃-C₈ heterocycle;

R¹⁰ is selected from the group consisting of: C₁-C₆ alkyl, C₂-C₆ alkenyl, C₃-C₈ cycloalkyl, and C₃-C₈ heterocycle;

R¹¹ and R¹² are each independently selected from the group consisting of: C₁-C₆ alkyl, C₂-C₆ alkenyl, C₃-C₈ cycloalkyl and C₃-C₈ heterocycle; or R₁₁ and R₁₂ are taken together with the nitrogen to which they are attached to form a 4-7 membered heterocycle,

or a pharmaceutically acceptable prodrug thereof.

In some embodiments, the salt is a salt of Formula I(c):

[chemical structure]

wherein:

X⁻ is a pharmaceutically acceptable anion;

R¹, R², R³, R⁴, R⁵, R⁶, R⁷, R⁸, and R⁹ are as defined above;

L¹ and L² are each independently C₁-C₆ alkylene or C₂-C₆ alkenylene; or L¹ and L² are taken together with the nitrogen to which they are attached to form a bridged heterocyclic moiety;

L⁴ is a direct bond, a C₁-C₆ alkylene, or a C₂-C₆ alkenylene;

R¹² is selected from the group consisting of hydrogen, C₁-C₆ alkyl, C₂-C₆ alkenyl, C₃-C₈ cycloalkyl, and C₃-C₈ heterocycle;

R¹³ is selected from the group consisting of C₁-C₆ alkyl, C₂-C₆ alkenyl, C₃-C₈ cycloalkyl, and C₃-C₈ heterocycle;

R¹⁴ and R¹⁵ are each independently selected from the group consisting of C₁-C₆ alkyl, C₂-C₆ alkenyl, C₃-C₈ cycloalkyl, and C₃-C₈ heterocycle; or R¹⁴ and R¹⁵ are taken together with the nitrogen to which they are attached to form a 4-7 membered heterocycle,

or a pharmaceutically acceptable prodrug thereof.

In some embodiments, the salt is a salt of Formula I(d):

[chemical structure]

wherein:

X⁻ is a pharmaceutically acceptable anion;

R¹, R², R³, R⁴, R⁵, R⁶, R⁷, R⁸, and R⁹ are as defined above;

L⁵, L⁶, L⁷, and L⁸ are each independently C₁-C₄ alkylene;

R¹⁰ is selected from the group consisting of: C₁-C₆ alkyl, C₂-C₆ alkenyl, C₃-C₈ cycloalkyl, and C₃-C₈ heterocycle;

R¹¹ is selected from the group consisting of C₁-C₆ alkyl, C₂-C₆ alkenyl, C₃-C₈ cycloalkyl, and C₃-C₈ heterocycle, wherein said alkyl, alkenyl, or cycloalkyl groups may be unsubstituted or substituted with an aryl such as phenyl,

or a pharmaceutically acceptable prodrug thereof.

In some embodiments, the salt is a salt of Formula I(e):

[chemical structure]

wherein:

X⁻ is a pharmaceutically acceptable anion;

R¹, R², R³, R⁴, R⁵, R⁶, R⁷, R⁸, and R⁹ are as defined above;

L¹ and L² are each independently C₁-C₆ alkylene or alkenylene;

R¹² and R¹⁶ are each independently selected from the group consisting of: hydrogen, C₁-C₆ alkyl, C₂-C₆ alkenyl, C₃-C₈ cycloalkyl, and C₃-C₈ heterocycle;

R¹⁰ is selected from the group consisting of: C₁-C₆ alkyl, C₂-C₆ alkenyl, C₃-C₈ cycloalkyl, and C₃-C₈ heterocycle; and

R¹¹ is selected from the group consisting of C₁-C₆ alkyl, C₂-C₆ alkenyl, C₃-C₈ cycloalkyl, and C₃-C₈ heterocycle, wherein said alkyl, alkenyl, or cycloalkyl groups may be unsubstituted or substituted with an aryl such as phenyl,

or a pharmaceutically acceptable prodrug thereof.

In some embodiments of the above-noted formulas, X⁻ is a halide such as, Br⁻ or I⁻.

Also provided herein is an amine oxide of Formula II:

[chemical structure]

wherein:

one of either R¹⁰ or R¹¹ is oxygen, and the other is selected from the group consisting of: C₁-C₆ alkyl, C₂-C₆ alkenyl, C₃-C₈ cycloalkyl, and C₃-C₈ heterocycle;

L¹ and L² are each independently C₁-C₆ alkylene or C₂-C₆ alkenylene;

R¹ is hydrogen, hydroxy, alkoxy, C₁₋₆ alkylester, or alkyl;

R² is H or alkyl;

R³ is H, alkyl, or hydroxy;

R⁴, R⁵ and R⁸ are each independently alkyl (e.g., methyl, ethyl, propyl);

R⁶ and R⁷ are each independently selected from the group consisting of H, hydroxy, and alkyl (e.g., methyl, ethyl, propyl); and

R⁹ is hydroxy or oxo;

or a pharmaceutically acceptable prodrug thereof,

wherein said alkylene, alkenylene, alkyl, alkenyl, cycloalkyl, and heterocycle groups may each independently be unsubstituted or substituted (e.g., 1-3 times).

Further provided is a quaternary ammonium salt of Formula III:

[chemical structure]

wherein:

X⁻ is a pharmaceutically acceptable anion;

L¹ and L² are each independently C₁-C₆ alkylene or C₂-C₆ alkenylene; or L¹ and L² are taken together with the two nitrogens to which they are attached to form a bridged heterocycle;

R¹⁰ is selected from the group consisting of: C₁-C₆ alkyl, C₂-C₆ alkenyl, C₃-C₈ cycloalkyl, and C₃-C₈ heterocycle;

R¹¹ is selected from the group consisting of: C₁-C₆ alkyl, C₂-C₆ alkenyl, C₃-C₈ cycloalkyl, and C₃-C₈ heterocycle; wherein said alkyl, alkenyl, or cycloalkyl groups may be unsubstituted or substituted with an aryl such as phenyl;

R² is H or alkyl;

R³ is H, alkyl, or hydroxy;

R⁴, R⁵ and R⁸ are each independently alkyl (e.g., methyl, ethyl, propyl);

R⁶, R⁷ and R⁹ are each independently selected from the group consisting of H, hydroxy, and alkyl (e.g., methyl, ethyl, propyl);

or a pharmaceutically acceptable prodrug thereof.

In some embodiments, X⁻ is a halide such as, Br⁻ or I⁻.

Also provided is an amine oxide of Formula IV:

[chemical structure]

wherein:

L¹ and L² are each independently C₁-C₆ alkylene or C₂-C₆ alkenylene; or L¹ and L² are taken together with the two nitrogens to which they are attached to form a bridged heterocyclic moiety;

one of either R¹⁰ or R¹¹ is oxygen, and the other is selected from the group consisting of: C₁-C₆ alkyl, C₂-C₆ alkenyl, C₃-C₈ cycloalkyl, and C₃-C₈ heterocycle;

R¹ is hydrogen, hydroxy, alkoxy, C₁₋₆ alkyl ester, or alkyl;

R² and R⁴ are each independently H or alkyl;

R³ is H, alkyl, or hydroxy;

R⁵ is an alkyl (such as methyl);

R⁶ and R⁷ are each independently selected from the group consisting of H, hydroxy, and alkyl (e.g., methyl, ethyl, propyl);

Y is O or N(R¹⁷)(R¹⁸);

R¹⁷ is selected from the group consisting of: hydrogen, C₁-C₆ alkyl, C₂-C₆ alkenyl, C₃-C₈ cycloalkyl, C₃-C₈ heterocyclyl, and —(C═O)—N(R¹⁸)(R¹⁹);

R¹⁸ and R¹⁹ are independently selected from the group consisting of: hydrogen, C₁-C₆ alkyl, C₂-C₆ alkenyl, C₃-C₈ cycloalkyl, and C₃-C₈ heterocyclyl,

or a pharmaceutically acceptable prodrug thereof,

wherein said alkylene, alkenylene, alkyl, alkenyl, cycloalkyl, and heterocycle groups may each independently be unsubstituted or substituted (e.g., 1-3 times).

Also provided is a pharmaceutical composition including the salt or amine oxide as described herein, or a pharmaceutically acceptable prodrug thereof, and a pharmaceutically acceptable carrier. In some embodiments, the composition is formulated for intravenous, oral, subcutaneous, or intramuscular administration.

Further provided is a method of treating cancer in a subject in need thereof, comprising administering to the subject an effective amount of a salt or amine oxide as taught herein, or a pharmaceutically acceptable prodrug thereof. In some embodiments, the cancer is myelodysplastic syndrome, chronic lymphocytic leukemia, chronic myelomonocytic leukemia, acute myeloid leukemia, colon cancer, pancreatic cancer, endometrial cancer, ovarian cancer or breast cancer. In some embodiments, the subject has cancer that is positive for mutations in the Splicing factor 3B subunit 1 (SF3B1) protein.

Still further provided is the use of a salt or amine oxide as described herein, or a pharmaceutically acceptable prodrug thereof, in a method of therapeutic treatment. In some embodiments, the therapeutic treatment is for the treatment of cancer. In some embodiments, the cancer is myelodysplastic syndrome, chronic lymphocytic leukemia, chronic myelomonocytic leukemia, acute myeloid leukemia, colon cancer, pancreatic cancer, endometrial cancer, ovarian cancer or breast cancer, or any subset thereof. In some embodiments, the cancer is positive for mutations in the Splicing factor 3B subunit 1 (SF3B1) protein.

Also provided is the use of a salt or amine oxide as taught herein, or a pharmaceutically acceptable prodrug thereof, in the preparation of a medicament. In some embodiments, the medicament is for the treatment of cancer. In some embodiments, the cancer is myelodysplastic syndrome, chronic lymphocytic leukemia, chronic myelomonocytic leukemia, acute myeloid leukemia, colon cancer, pancreatic cancer, endometrial cancer, ovarian cancer or breast cancer, or any subset thereof. In some embodiments, the cancer is positive for mutations in the Splicing factor 3B subunit 1 (SF3B1) protein.

### DETAILED DESCRIPTION OF CERTAIN EMBODIMENTS

### A. Definitions

Compounds as active agents of this invention include those described generally above, and are further illustrated by the embodiments, sub-embodiments, and species disclosed herein. As used herein, the following definitions shall apply unless otherwise indicated.

As described herein, compounds of the invention may optionally be substituted with one or more substituents, such as those illustrated generally above, or as exemplified by particular classes, subclasses, and species of the invention. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

“Isomers” refer to compounds having the same number and kind of atoms and hence the same molecular weight, but differing with respect to the arrangement or configuration of the atoms.

“Stereoisomers” refer to isomers that differ only in the arrangement of the atoms in space.

“Diastereoisomers” refer to stereoisomers that are not mirror images of each other.

“Enantiomers” refers to stereoisomers that are non-superimposable mirror images of one another.

Enantiomers include “enantiomerically pure” isomers that comprise substantially a single enantiomer, for example, greater than or equal to 90%, 92%, 95%, 98%, or 99%, or equal to 100% of a single enantiomer.

“Stereomerically pure” as used herein means a compound or composition thereof that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of diastereomers, and substantially free of the opposite enantiomer, of the compound. A typical stereomerically pure compound comprises greater than about 80% by weight of one stereoisomer of the compound and less than about 20% by weight of the other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. See, e.g., U.S. Pat. No. 7,189,715.

“R” and “S” as terms describing isomers are descriptors of the stereochemical configuration at an asymmetrically substituted carbon atom. The designation of an asymmetrically substituted carbon atom as “R” or “S” is done by application of the Cahn-Ingold-Prelog priority rules, as are well known to those skilled in the art, and described in the International Union of Pure and Applied Chemistry (IUPAC) Rules for the Nomenclature of Organic Chemistry. Section E, Stereochemistry.

“Enantiomeric excess” (ee) of an enantiomer is [(the mole fraction of the major enantiomer) minus (the mole fraction of the minor enantiomer)]×100.

“Stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

“Amine oxide” or “amine-N-oxide” or “N-oxide” is a chemical compound that contains the functional group R³N⁺—O⁻, an N—O bond with three additional hydrogen and/or hydrocarbon sidechains attached to N. Sometimes it is written as R³N→O.

“Ar” or “aryl” refer to an aromatic carbocyclic moiety having one or more closed rings, Examples include, without limitation, phenyl, naphthyl, anthracenyl, phenanthracenyl, biphenyl, and pyrenyl.

“Heteroaryl” refers to a cyclic moiety having one or more closed rings, with one or more heteroatoms (oxygen, nitrogen or sulfur) in at least one of the rings, wherein at least one of the rings is aromatic, and wherein the ring or rings may independently be fused, and/or bridged. Examples include without limitation quinolinyl, isoquinolinyl, indolyl, furyl, thienyl, pyrazolyl, quinoxalinyl, pyrrolyl, indazolyl, thieno[2,3-c]pyrazolyl, benzofuryl, pyrazolo[1,5-a]pyridyl, thiophenylpyrazolyl, benzothienyl, benzothiazolyl, thiazolyl, 2-phenylthiazolyl, and isoxazolyl.

“Alkyl” or “alkyl group,” as used herein, means a straight-chain (i.e., unbranched), branched, or cyclic hydrocarbon chain that is completely saturated. In certain embodiments, alkyl groups contain 1-8 carbon atoms. In certain embodiments, alkyl groups contain 1-4 carbon atoms. In certain embodiments, alkyl groups contain 1-3 carbon atoms. In still other embodiments, alkyl groups contain 2-3 carbon atoms, and in yet other embodiments alkyl groups contain 1-2 carbon atoms. In certain embodiments, the term “alkyl” or “alkyl group” refers to a cycloalkyl group, also known as carbocycle. Non-limiting examples of exemplary alkyl groups include methyl, ethyl, propyl, isopropyl, butyl, cyclopropyl and cyclohexyl.

“Alkenyl” or “alkenyl group,” as used herein, refers to a straight-chain (i.e., unbranched), branched, or cyclic hydrocarbon chain that has one or more double bonds. In certain embodiments, alkenyl groups contain 2-8 carbon atoms. In certain embodiments, alkenyl groups contain 2-6 carbon atoms. In still other embodiments, alkenyl groups contain 3-4 carbon atoms, and in yet other embodiments alkenyl groups contain 2-3 carbon atoms. According to another aspect, the term alkenyl refers to a straight chain hydrocarbon having two double bonds, also referred to as “diene.” In other embodiments, the term “alkenyl” or “alkenyl group” refers to a cycloalkenyl group. Non-limiting examples of exemplary alkenyl groups include —CH═CH₂, —CH₂CH═CH₂ (also referred to as allyl), —CH═CHCH₃, —CH₂CH₂CH═CH₂, —CH₂CH═CHCH₃, —CH═CHCH₂CH₃, —CH═CHCH═CH₂, and cyclobutenyl.

“Alkoxy”, or “alkylthio”, as used herein, refers to an alkyl group, as previously defined, attached to the principal carbon chain through an oxygen (“alkoxy”) or sulfur (“alkylthio”) atom.

“Alkylene” refers to a saturated straight or branched chain alkyl group having two or more points of attachments. In some embodiments, alkylene has from one to six carbon atoms, for example, methylene, ethylene, propylene, n-butylene and the like. Examples of alkylene having two points of attachment include, but are not limited to, methylene (—CH₂—), ethylene (—CH₂CH₂—), propylene (—CH₂CH₂CH₂—), and dimethylpropylene (—CH₂C(CH₃)₂CH₂—). Examples of a branched alkylene which may have three points of attachment include, but are not limited to, isobutylene (—CH₂C(CH₂—)CH₂—):

[chemical structure]

“Alkenylene” refers to an unsaturated straight or branched chain alkenyl group having one or more double bonds formed by mono or dialkenyl substitution of alkylene. In some embodiments, an alkenylene group has 2-6 carbon atoms. In other embodiments, an alkenylene group has 2-6, 2-5, 2-4, or 2-3 carbon atoms. In some embodiments, an alkenylene has two double bonds. Exemplary alkenylene groups include, but are not limited to, ethenylene (—CH═CH—), propenylene (—CH═CHCH₂— or —CH₂CH═CH—), butenylene (—CH═CHCH₂CH₂—, —CH₂CH═CHCH₂—, or —CH₂CH₂CH═CH—). Unless otherwise indicated, each alkenylene group can be in the cis or trans configuration.

“C₁₋₆ alkyl ester or amide” refers to a C₁₋₆ alkyl ester or a C₁₋₆ alkyl amide where each C₁₋₆ alkyl group is as defined above. Such C₁₋₆ alkyl ester groups are of the formula (C₁₋₆ alkyl)OC(═O)— or (C₁₋₆ alkyl)C(═O)O—. Such C₁₋₆ alkyl amide groups are of the formula (C₁₋₆ alkyl)NHC(═O)— or (C₁₋₆ alkyl)C(═O)NH—.

“C₂₋₆ alkenyl ester or amide” refers to a C₂₋₆ alkenyl ester or a C₂₋₆ alkenyl amide where each C₂₋₆ alkenyl group is as defined above. Such C₂₋₆ alkenyl ester groups are of the formula (C₂₋₆ alkenyl)OC(═O)— or (C₂₋₆ alkenyl)C(═O)O—. Such C₂₋₆ alkenyl amide groups are of the formula (C₂₋₆ alkenyl)NHC(═O)— or (C₂₋₆ alkenyl)C(═O)NH—.

“Fluoromethyl” as used herein refers to a methyl group substituted with one or more fluoro atoms (e.g., monofluoromethyl, difluoromethyl, trifluoromethyl).

“Fluoromethoxy” as used herein, refers to a fluoromethyl group, as previously defined, attached to the principal carbon chain through an oxygen atom.

“Heteroatom” refers to O, S or N.

“Heterocycle” or “heterocyclic” as used herein, means a monocyclic heterocycle, a bicyclic heterocycle, or a tricyclic heterocycle containing at least one heteroatom in the ring.

The monocyclic heterocycle is a 3-, 4-, 5-, 6-, 7, or 8-membered ring containing at least one heteroatom independently selected from the group consisting of O, N, and S. In some embodiments, the heterocycle is a 3- or 4-membered ring containing one heteroatom selected from the group consisting of O, N and S. In some embodiments, the heterocycle is a 5-membered ring containing zero or one double bond and one, two or three heteroatoms selected from the group consisting of O, N and S. In some embodiments, the heterocycle is a 6-, 7-, or 8-membered ring containing zero, one or two double bonds and one, two or three heteroatoms selected from the group consisting of O, N and S. Representative examples of monocyclic heterocycle include, but are not limited to, azetidinyl, azepanyl, aziridinyl, diazepanyl, 1,3-dioxanyl, 1,3-dioxolanyl, dihydropyranyl (including 3,4-dihydro-2H-pyran-6-yl), 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydropyranyl, tetrahydropyranyl (including tetrahydro-2H-pyran-4-yl), tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

The bicyclic heterocycles of the present invention are exemplified by a monocyclic heterocycle fused to an aryl group, or a monocyclic heterocycle fused to a monocyclic cycloalkyl, or a monocyclic heterocycle fused to a monocyclic cycloalkenyl, or a monocyclic heterocycle fused to a monocyclic heterocycle. Representative examples of bicyclic heterocycles include, but are not limited to, 3,4-dihydro-2H-pyranyl, 1,3-benzodioxolyl, 1,3-benzodithiolyl, 2,3-dihydro-1,4-benzodioxinyl, 2,3-dihydro-1-benzofuranyl, 2,3-dihydro-1-benzothienyl, 2,3-dihydro-1H-indolyl, and 1,2,3,4-tetrahydroquinolinyl.

In certain embodiments, the bicyclic heterocycle is a spiro heterocycle. As known in the art, a “spiro” heterocycle is a bicyclic moiety with rings connected through just one atom. The connecting atom is also called the spiro atom and most often is a quaternary atom such as carbon or nitrogen. Spiro compounds may be designated with the infix spiro followed by square brackets containing the number of atoms in the smaller ring and the number of atoms in the larger ring excluding the spiroatom itself; the numbers being separated by a dot. Example of such compounds include, but are not limited to, 2,6-diazaspiro[3.3]heptane.

The tricyclic heterocycle is a bicyclic heterocycle fused to an aryl group, or a bicyclic heterocycle fused to a monocyclic cycloalkyl, or a bicyclic heterocycle fused to a monocyclic cycloalkenyl, or a bicyclic heterocycle fused to a monocyclic heterocycle. Representative examples of tricyclic heterocycles include, but are not limited to, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b,d]thienyl.

The heterocycle groups of the present invention are connected to the parent molecular moiety through any substitutable carbon atom or any substitutable nitrogen, oxygen or sulfur atom contained within the groups and may contain one or two alkylene bridges of 1, 2, 3, or 4 carbon atoms, each linking two non-adjacent carbon atoms of the groups. Examples of such “bridged” heterocycle groups include, but are not limited to, oxatricyclo[3.3.1.1^{3,7}]decyl (including 2-oxatricyclo[3.3.1.1^{3,7}]decyl), 2,4-dioxabicyclo[4.2.1]nonyl, oxabicyclo[2.2.1]heptyl (including 2-oxabicyclo[2.2.1]heptyl) and 2,5diazabicyclo[2.2.1]heptane.

In the above heteroaryl and heterocycles the nitrogen or sulfur atoms can be optionally oxidized to various oxidation states. In a specific example, the group S(O)₀₋₂ refers to —S-(sulfide), —S(O)-(sulfoxide), and —SO₂-(sulfone) respectively. For convenience, nitrogens, particularly but not exclusively, those defined as annular aromatic nitrogens, are meant to include those corresponding N-oxide forms. Thus, for a compound of the invention having, for example, a pyridyl ring; the corresponding pyridyl-N-oxide is meant to be included as another compound of the invention.

“Treatment,” “treat,” and “treating” refer to reversing, alleviating, delaying the onset of, inhibiting the progress of, or preventing a disease or disorder as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

“Patient” or “subject”, as used herein, means an animal subject, preferably a mammalian subject (e.g., dog, cat, horse, cow, sheep, goat, monkey, etc.), and particularly human subjects (including both male and female subjects, and including neonatal, infant, juvenile, adolescent, adult and geriatric subjects).

“Pharmaceutically acceptable carrier” as used herein refers to a nontoxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, cyclodextrins, sodium carboxymethylcellulose, polyacrylates, waxes, polyethylene-polyoxypropylene-block polymers, polyethylene glycol and wool fat.

“Pharmaceutically acceptable anion” refers to an anion of the compound of the invention, which compound possesses the desired pharmacological activity and is neither biologically nor otherwise undesirable. Examples of anions include, but are not limited to, halides such as iodide, bromide, chloride, fluoride, or other anions such as acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, formate, fumarate, glucoheptanoate, glycerophosphate, hemisulfate, heptanoate, hexanoate, 2-hydroxyethane-sulfonate, lactate, maleate, methanesulfonate, 2-naphthalenesulfonate, nicotinate, oxalate, phosphate, sulfate, tartrate, thiocyanate, tosylate and undecanoate. See, e.g., Haynes et al., “Commentary: Occurrance of Pharmaceutically Acceptable Anions and Cations in the Cabridge Structural Database,” J. Pharmaceutical Sciences, vol. 94, no. 10 (2005), which are incorporated by reference herein. In some embodiments, the basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as phenethyl bromides. In other embodiments, the basic nitrogen-containing groups can be quarternized to an amine oxide with an oxidizing agent including, but not limited to, sodium metaperiodate.

“Pharmaceutically acceptable prodrug” as used herein refers to compounds that, upon administration, are subsequently converted in vivo to yield an active compound of the formulas of the present invention, for example, by normal metabolic processes or hydrolysis in blood, which active compounds have the desired pharmacological activity and which compounds are neither biologically nor otherwise undesirable. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

Unless indicated otherwise, nomenclature used to describe chemical groups or moieties as used herein follow the convention where, reading the name from left to right, the point of attachment to the rest of the molecule is at the right-hand side of the name. For example, the group “(C₁₋₃ alkoxy)C₁₋₃ alkyl,” is attached to the rest of the molecule at the alkyl end. Further examples include methoxyethyl, where the point of attachment is at the ethyl end, and methylamino, where the point of attachment is at the amine end.

Unless indicated otherwise, where a chemical group is described by its chemical formula, including two terminal bond moieties indicated by “—,” it will be understood that the attachment is read from left to right.

Unless otherwise stated, structures depicted herein are also meant to include all enantiomeric, diastereomeric, and geometric (or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### B. Compounds

Provided herein according to some embodiments is a quaternary salt of Formula I:

[chemical structure]

wherein:

Y is a quaternary nitrogen;

X⁻ is a pharmaceutically acceptable anion;

L¹ is alkylene or alkenylene; or L¹ and R¹² are joined together to form a branched alkylene or alkenylene;

L² is H, alkyl; or L² and R¹² are joined together to form an alkylene or alkenylene; or L² and L¹ are joined together to form a branched alkylene or alkenylene;

or L¹, L² and R¹² are joined together, with the two nitrogens to which they are attached, to form a spiro heterocycle or a bridged heterocycle;

R¹² is alkyl, or R¹² is joined to L¹ and/or L²;

R¹⁰ and R¹¹ are each independently: alkyl, alkenyl, cycloalkyl, aryl, arylalkyl, arylalkenyl, and heterocycle; or R¹⁰ and R¹¹ are taken together with the nitrogen to which they are attached to form a heterocycle;

R¹ is hydrogen, hydroxy, alkoxy, C₁₋₆ alkyl ester or alkyl;

R² is H or alkyl;

R³ is H or alkyl, or hydroxy;

R⁴, R⁵ and R⁸ are each independently alkyl (e.g., methyl, ethyl, propyl);

R⁶ and R⁷ are each independently selected from the group consisting of H, hydroxy, and alkyl (e.g., methyl, ethyl, propyl); and

R⁹ is hydroxy or oxo;

or a pharmaceutically acceptable prodrug thereof,

wherein said alkylene, alkenylene, alkyl, alkenyl, cycloalkyl, aryl, arylalkyl, arylalkenyl, and heterocycle groups may each independently be unsubstituted or substituted (e.g., 1-3 times).

In some embodiments, the salt is a salt of Formula I(a):

[chemical structure]

wherein:

X⁻ is a pharmaceutically acceptable anion;

R¹, R², R³, R⁴, R⁵, R⁶, R⁷, R⁸, and R⁹ are as defined above;

L¹ and L² are each independently C₁-C₆ alkylene or C₂-C₆ alkenylene; or L¹ and L² are taken together with the two nitrogens to which they are attached to form a bridged heterocyclic moiety;

R¹⁰ and R¹¹ are each independently selected from the group consisting of: C₁-C₆ alkyl, C₂-C₆ alkenyl, C₃-C₈ cycloalkyl, and C₃-C₈ heterocycle; or R₁₀ and R₁₁ are taken together with the nitrogen to which they are attached to form a 5-7 membered heterocycle;

or a pharmaceutically acceptable prodrug thereof.

In some embodiments, the salt is a salt of Formula I(b):

[chemical structure]

wherein:

X⁻ is a pharmaceutically acceptable anion;

R¹, R², R³, R⁴, R⁵, R⁶, R⁷, R⁸, and R⁹ are as defined above;

L¹ is C₁-C₆ alkylene or C₂-C₆ alkenylene;

R¹³ is selected from the group consisting of: hydrogen, C₁-C₆ alkyl, C₂-C₆ alkenyl, C₃-C₈ cycloalkyl, and C₃-C₈ heterocycle;

R¹⁰ is selected from the group consisting of: C₁-C₆ alkyl, C₂-C₆ alkenyl, C₃-C₈ cycloalkyl, and C₃-C₈ heterocycle;

R¹¹ and R¹² are each independently selected from the group consisting of: C₁-C₆ alkyl, C₂-C₆ alkenyl, C₃-C₈ cycloalkyl and C₃-C₈ heterocycle; or R₁₁ and R₁₂ are taken together with the nitrogen to which they are attached to form a 4-7 membered heterocycle,

or a pharmaceutically acceptable prodrug thereof.

In some embodiments, the salt is a salt of Formula I(c):

[chemical structure]

wherein:

X⁻ is a pharmaceutically acceptable anion; R¹, R², R³, R⁴, R⁵, R⁶, R⁷, R⁸, and R⁹ are as defined above;

L¹ and L² are each independently C₁-C₆ alkylene or C₂-C₆ alkenylene; or L¹ and L² are taken together with the nitrogen to which they are attached to form a bridged heterocyclic moiety;

L⁴ is a direct bond, or a C₁-C₆ alkylene, or a C₂-C₆ alkenylene;

R¹² is selected from the group consisting of hydrogen, C₁-C₆ alkyl, C₂-C₆ alkenyl, C₃-C₈ cycloalkyl, and C₃-C₈ heterocycle;

R¹³ is selected from the group consisting of C₁-C₆ alkyl, C₂-C₆ alkenyl, C₃-C₈ cycloalkyl, and C₃-C₈ heterocycle;

R¹⁴ and R¹⁵ are each independently selected from the group consisting of C₁-C₆ alkyl, C₂-C₆ alkenyl, C₃-C₈ cycloalkyl, and C₃-C₈ heterocycle; or R¹⁴ and R¹⁵ are taken together with the nitrogen to which they are attached to form a 4-7 membered heterocycle,

or a pharmaceutically acceptable prodrug thereof.

In some embodiments, the salt is a salt of Formula I(d):

[chemical structure]

wherein:

X⁻ is a pharmaceutically acceptable anion;

R¹, R², R³, R⁴, R⁵, R⁶, R⁷, R⁸, and R⁹ are as defined above;

L⁵, L⁶, L⁷, and L⁸ are each independently C₁-C₄ alkylene;

R¹⁰ is selected from the group consisting of: C₁-C₆ alkyl, C₂-C₆ alkenyl, C₃-C₈ cycloalkyl, and C₃-C₈ heterocycle;

R¹¹ is selected from the group consisting of C₁-C₆ alkyl, C₂-C₆ alkenyl, C₃-C₈ cycloalkyl, and C₃-C₈ heterocycle, wherein said alkyl, alkenyl, or cycloalkyl groups may be unsubstituted or substituted with an aryl such as phenyl,

or a pharmaceutically acceptable prodrug thereof.

In some embodiments, the salt is a salt of Formula I(e):

[chemical structure]

wherein:

X⁻ is a pharmaceutically acceptable anion;

R¹, R², R³, R⁴, R⁵, R⁶, R⁷, R⁸, and R⁹ are as defined above;

L¹ and L² are each independently C₁-C₆ alkylene or alkenylene;

R¹² and R¹⁶ are each independently selected from the group consisting of: hydrogen, C₁-C₆ alkyl, C₂-C₆ alkenyl, C₃-C₈ cycloalkyl, and C₃-C₈ heterocycle;

R¹⁰ is selected from the group consisting of: C₁-C₆ alkyl, C₂-C₆ alkenyl, C₃-C₈ cycloalkyl, and C₃-C₈ heterocycle; and

R¹¹ is selected from the group consisting of C₁-C₆ alkyl, C₂-C₆ alkenyl, C₃-C₈ cycloalkyl, and C₃-C₈ heterocycle, wherein said alkyl, alkenyl, or cycloalkyl groups may be unsubstituted or substituted with an aryl such as phenyl,

or a pharmaceutically acceptable prodrug thereof.

In some embodiments of the above-noted formulas, X⁻ is a halide such as, Br⁻ or I⁻.

Also provided herein is an amine oxide of Formula II:

[chemical structure]

wherein:

one of either R¹⁰ or R¹¹ is oxygen, and the other is selected from the group consisting of: C₁-C₆ alkyl, C₂-C₆ alkenyl, C₃-C₈ cycloalkyl, and C₃-C₈ heterocycle;

L¹ and L² are each independently C₁-C₆ alkylene or C₂-C₆ alkenylene;

R¹ is hydrogen, hydroxy, alkoxy, C₁₋₆ alkyl ester, or alkyl;

R² is H or alkyl;

R³ is H, alkyl, or hydroxy;

R⁴, R⁵ and R⁸ are each independently alkyl (e.g., methyl, ethyl, propyl);

R⁶ and R⁷ are each independently selected from the group consisting of H, hydroxy, and alkyl (e.g., methyl, ethyl, propyl); and

R⁹ is hydroxy or oxo;

or a pharmaceutically acceptable prodrug thereof,

wherein said alkylene, alkenylene, alkyl, alkenyl, cycloalkyl, and heterocycle groups may each independently be unsubstituted or substituted (e.g., 1-3 times).

Further provided is a quaternary ammonium salt of Formula III:

[chemical structure]

wherein:

X⁻ is a pharmaceutically acceptable anion;

L¹ and L² are each independently C₁-C₆ alkylene or C₂-C₆ alkenylene; or L¹ and L² are taken together with the two nitrogens to which they are attached to form a bridged heterocycle;

R¹⁰ is selected from the group consisting of: C₁-C₆ alkyl, C₂-C₆ alkenyl, C₃-C₈ cycloalkyl, and C₃-C₈ heterocycle;

R¹¹ is selected from the group consisting of: C₁-C₆ alkyl, C₂-C₆ alkenyl, C₃-C₈ cycloalkyl, and C₃-C₈ heterocycle; wherein said alkyl, alkenyl, or cycloalkyl groups may be unsubstituted or substituted with an aryl such as phenyl;

R² is H or alkyl;

R³ is H, alkyl, or hydroxy;

R⁴, R⁵ and R⁸ are each independently alkyl (e.g., methyl, ethyl, propyl);

R⁶, R⁷ and R⁹ are each independently selected from the group consisting of H, hydroxy, and alkyl (e.g., methyl, ethyl, propyl);

or a pharmaceutically acceptable prodrug thereof.

In some embodiments, X⁻ is a halide such as, Br⁻ or I⁻.

Also provided is an amine oxide of Formula IV:

[chemical structure]

wherein:

L¹ and L² are each independently C₁-C₆ alkylene or C₂-C₆ alkenylene; or L¹ and L² are taken together with the two nitrogens to which they are attached to form a bridged heterocyclic moiety;

one of either R¹⁰ or R¹¹ is oxygen, and the other is selected from the group consisting of: C₁-C₆ alkyl, C₂-C₆ alkenyl, C₃-C₈ cycloalkyl, and C₃-C₈ heterocycle;

R¹ is hydrogen, hydroxy, alkoxy, C₁₋₆ alkyl ester or alkyl;

R² and R⁴ are each independently H or alkyl;

R³ is H, alkyl, or hydroxy;

R⁵ is an alkyl such as methyl;

R⁶ and R⁷ are each independently selected from the group consisting of H, hydroxy, and alkyl (e.g., methyl, ethyl, propyl);

Y is O or N(R¹⁷)(R¹⁸);

R¹⁷ is selected from the group consisting of: hydrogen, C₁-C₆ alkyl, C₂-C₆ alkenyl, C₃-C₈ cycloalkyl, C₃-C₈ heterocyclyl, and —(C═O)—N(R¹⁸)(R¹⁹);

R¹⁸ and R¹⁹ are independently selected from the group consisting of: hydrogen, C₁-C₆ alkyl, C₂-C₆ alkenyl, C₃-C₈ cycloalkyl, and C₃-C₈ heterocyclyl,

or a pharmaceutically acceptable prodrug thereof,

wherein said alkylene, alkenylene, alkyl, alkenyl, cycloalkyl, and heterocycle groups may each independently be unsubstituted or substituted (e.g., 1-3 times).

### C. Pharmaceutical Formulations

Active compounds of the present invention can be combined with a pharmaceutically acceptable carrier to provide pharmaceutical formulations thereof. The particular choice of carrier and formulation will depend upon the particular route of administration for which the composition is intended.

The compositions of the present invention may be suitable for parenteral, oral, inhalation spray, topical, rectal, nasal, buccal, vaginal or implanted reservoir administration, etc. The term “parenteral” as used herein includes subcutaneous, intravenous, intramuscular, intra-articular, intra-synovial, intrasternal, intrathecal, intrahepatic, intralesional and intracranial injection or infusion techniques. In particular embodiments, the compounds are administered intravenously, orally, subcutaneously, or via intramuscular administration. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a nontoxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

For oral administration, the active compounds may be provided in an acceptable oral dosage form, including, but not limited to, capsules, tablets, aqueous suspensions or solutions. In the case of tablets for oral use, carriers commonly used include lactose and corn starch. Lubricating agents, such as magnesium stearate, may also be added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

### D. Subjects and Methods of Use

Compounds of the present invention may be used to treat various types of cancers, including those responsive to agents that target the spliceosome, including SF3B1 spliceosome. As noted above, the anti-tumor activity of Pladienolide B has been connected to its targeting of the SF3b spliceosome, inhibiting splicing and altering the pattern of gene expression. (Kotake et al., “Splicing factor SF3b as a target of the antitumor natural product pladienolide,” Nature Chemical Biology 2007, 3, 570-575). Mutations in the Splicing factor 3B subunit 1 (SF3B1) protein of the spliceosome have been implicated in a number of cancers.

The following examples are illustrative of the various cancers responsive to agents that target the spliceosome, and are not meant to limit the scope of the invention in any way. Active compounds of the present invention may be administered to patients or subjects to treat a variety of different cancers or conditions, particularly patients or subjects afflicted with:

a) Myelodysplastic syndrome (MDS): See, e.g., “SF3B1 mutations in myelodysplastic syndromes: clinical associations and prognostic implications,” Damm F. et al. Leukemia, 2011, 1-4; “Frequent pathway mutations of splicing machinery in myelodysplasia,” Yoshida K. et al, Nature, 2011, 478, 64-69; “Clinical significance of SF3B1 mutations in myelodysplastic syndromes and myelodysplastic/myeloproliferative neoplasms,” Malcovati L. et al., Blood, 2011, 118, 24, 6239-6246; “Mutations in the spliceosome machinery, a novel and ubiquitous pathway in leukemogenesis,” Makishima et al, Blood, 2012, 119, 3203-3210; “Somatic SF3B1 mutation in myelodysplasia with ring sideroblasts,” Pappaemannuil, E. et al, New England J. Med. 2011, DOI 10.1056/NEJMoa1103283.

b) Chronic lymphocytic leukemia (CLL): See, e.g., “Defects in the spliceosomal machinery: a new pathway of leukaemogenesis,” Maciejewski, J. P., Padgett, R. A., Br. J. Haematology, 2012, 1-9; “Mutations in the SF3B1 splicing factor in chronic lymphocytic leukemia: associations with progression and fludarabine-refractoriness,” Rossi et al, Blood, 2011, 118, 6904-6908; “Exosome sequencing identifies recurrent mutations of the splicing factor SF3B1 gene in chronic lymphocytic leukemia,” Quesada et al, Nature Genetics, 2011, 44, 47-52.

c) Chronic myelomonocytic leukemia (CMML): See, e.g., Yoshida et al, Nature 2011; “Spliceosomal gene mutations are frequent events in the diverse mutational spectrum of chronic myelomonocytic leukemia but largely absent in juvenile myelomonocytic leukemia,” Kar S. A. et al, Haematologia, 2012, DOI: 10.3324/haematol.2012.064048.

d) Acute myeloid leukemia (AML): See, e.g., Malcovati et al., Blood 2011; Yoshida et al, Nature 2011.

e) Breast cancer: See, e.g., “Whole genome analysis informs breast cancer response to aromatase inhibition,” Ellis et al, Nature, 2012, 486, 353-360.

The active compounds are administered to the subjects in a treatment effective, or therapeutically effective, amount. The amount of the compounds of the present invention that may be combined with the carrier materials to produce a composition in a single dosage form will vary depending upon the subject treated and the particular route of administration. Preferably, the compositions should be formulated so that a dosage of between 0.01-100 mg/kg body weight/day of the active agent can be administered to a patient receiving these compositions. In certain embodiments, the compositions of the present invention provide a dosage of between 0.01 mg and 50 mg is provided. In other embodiments, a dosage of between 0.1 and 25 mg or between 5 mg and 40 mg is provided.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active agent of the present invention in the composition will also depend upon the particular compound/salt in the composition.

In some embodiments, subjects have been screened for and/or are positive for mutations in the Splicing factor 3B subunit 1 (SF3B1) protein, wherein the presence of mutations (“positive”) indicates the subject is responsive to a method of treatment comprising administration of an active compound targeting this protein and/or the spliceosome.

Screening for the mutations may be carried out by any known means, for example, genotyping, phenotyping, etc., by way of nucleic acid amplification, electrophoresis, microarrays, blot, functional assays, immunoassays, etc. Methods of screening may include, for example, collecting a biological sample from said subject containing the cancerous cells/tissue.

In order that the invention described herein may be more fully understood, the following examples are set forth. It should be understood that these examples are for illustrative purposes only and are not to be construed as limiting this invention in any manner.

### Examples 1-31

### General

Microwave heating was done using a Biotage Emrys Liberator or Initiator microwave. Column chromatography was carried out using an Isco Rf200d. Solvent removal was carried out using either a Büchi rotary evaporator or a Genevac centrifugal evaporator. Preparative LC/MS was conducted using a Waters autopurifier and 19×100 mm XTerra 5 micron MS C18 column under acidic mobile phase condition. NMR spectra were recorded using a Varian 400 MHz spectrometer.

When the term “inerted” is used to describe a reactor (e.g., a reaction vessel, flask, glass reactor, and the like) it is meant that the air in the reactor has been replaced with an essentially moisture-free or dry, inert gas (such as nitrogen, argon, and the like).

General methods and experimentals for preparing compounds of the present invention are set forth below. In certain cases, a particular compound is described by way of example. However, it will be appreciated that in each case a series of compounds of the present invention were prepared in accordance with the schemes and experimentals described below.

The following abbreviations are used herein:

### DEFINITIONS

The following abbreviations have the indicated meanings:

DMAP: 4-(Dimethylamino)pyridine

KHMDS: Potassium bis(trimethylsilyl)amide

LCMS: Liquid chromatography-mass spectrometry

TBAF: Tetrabutylammonium fluoride

TBSCl: tert-Butyldimethylsilyl chloride

TBSOTf: tert-Butyldimethylsilyl trifluoromethanesulfonate

TESCl: Chlorotriethylsilane

THF: Tetrahydrofuran

TLC: Thin-layer chromatography

PPTS: Pyridinium p-toluenesulfonate

Materials:

The following compounds are commercially available and/or can be prepared in a number of ways well known to one skilled in the art of organic synthesis. More specifically, disclosed compounds can be prepared using the reactions and techniques described herein. In the description of the synthetic methods described below, it is to be understood that all proposed reaction conditions, including choice of solvent, reaction atmosphere, reaction temperature, duration of the experiment, and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

U.S. Pat. Nos. 7,884,128 and 7,816,401, both entitled: Process for Total Synthesis of Pladienolide B and Pladienolide D, are incorporated by reference herein for synthesis of Pladienolide B and D.

### Synthetic Methods

### Analogs Originating from Pladienolide B

[chemical structure]

[chemical structure]

Step S-1: A solution of A (pladienolide B, 1.0 equiv.) in dichloromethane (0.2M) under nitrogen at room temperature was treated with DMAP (10.0 equiv.) and N,N-diisopropylethylamine (10.0 equiv.). The reaction was cooled to 0° C. and TESCl (10.0 equiv.) was added. The reaction was allowed to warm to room temperature and stirred for two days, or until the reaction was determined to be complete by LCMS or TLC. The reaction was then quenched with saturated sodium bicarbonate solution and the organic layer was separated, washed with brine, dried over magnesium sulfate, filtered, and concentrated in vacuo. The resulting oil was purified by silica gel column chromatography (gradient, hexanes/ethyl acetate as eluant) to afford the desired product (B).

Step. S-2: A solution of silyl ether B (1.0 equiv.) in toluene:THF (1:1, 0.04M) under nitrogen was treated with ethyl vinyl ether (40.0 equiv.) and PPTS (0.01 equiv.) at room temperature. The reaction was stirred for 16 hours, or until the reaction was determined to be complete by LCMS or TLC. Triethylamine (1.0 equiv.) was added and stirred for 5 minutes. The reaction was then diluted with methyl tert-butyl ether and washed with brine, dried over magnesium sulfate, filtered, and concentrated in vacuo. The resulting oil was purified by silica gel column chromatography (hexanes/ethyl acetate as eluant) to afford the desired product (C).

Step S-3: A solution of acetal C (1.0 equiv.) in MeOH (0.2M) under nitrogen at 0° C. was treated with sodium guanidine nitrate (1.1 equiv.) dropwise over three minutes. The reaction was allowed to warm to room temperature and stirred for 16 hours, or until the reaction is determined to be complete by LCMS or TLC. The reaction was then diluted with ethyl acetate and saturated ammonium chloride solution and the organic layer was washed with brine, dried over magnesium sulfate, filtered, and concentrated in vacuo. The resulting oil was purified by silica gel column chromatography (hexanes/ethyl acetate as eluant) to afford the desired product (D).

Step S-4: A solution of alcohol D (1.0 equiv.) in dichloromethane (0.05M) under nitrogen at room temperature was treated with DMAP (1.2 equiv.), triethylamine (5.0 equiv.), and 4-nitrophenylchloroformate (2.5 equiv.). The reaction was stirred for 16 hours, or until the reaction was determined to be complete by LCMS or TLC. The reaction was diluted with dichloromethane and washed with brine, dried over magnesium sulfate, filtered, and concentrated in vacuo. The resulting oil was purified by silica gel column chromatography (hexanes/ethyl acetate as eluant) to afford the desired product (E).

Step S-5: A solution of carbonate E (1.0 equiv.) in methyl tert-butyl ether (0.01M) under nitrogen at room temperature was treated with N,N-diisopropylethylamine (10 equiv.) and amine (see table). The reaction was stirred for 16 hours, or until the reaction was determined to be complete by LCMS or TLC. The reaction was diluted with methyl tert-butyl ether and washed with saturated ammonium bicarbonate solution, brine, dried over magnesium sulfate, filtered, and concentrated in vacuo. The resulting oil was purified by silica gel column chromatography (hexanes/ethyl acetate as eluant) to afford the desired product (F).

Step S-6: A solution of carbamate F (1.0 equiv.) in MeOH (0.01M) under nitrogen at room temperature was treated with PPTS (5.0 equiv.) The reaction was stirred for 2 hours, or until the reaction was determined to be complete by LCMS or TLC. The reaction was diluted with dichloromethane and washed with brine, dried over magnesium sulfate, filtered, and concentrated in vacuo. The resulting oil was purified by silica gel column chromatography (dichloromethane/methanol as eluant) to afford the desired product (G).

Step S-7: A solution of carbamate G (1.0 equiv.) in dichloromethane (0.01M) under nitrogen at room temperature was treated with methyl iodide (100 equiv.). The reaction was stirred for 4 hours, or until the reaction was determined to be complete by LCMS or TLC. The reaction was concentrated in vacuo and purified by silica gel column chromatography (dichloromethane/methanol as eluant) to afford the desired product H (Compounds 1-16, Table 1).

Examples 1-16 were prepared using the above scheme.

TABLE 1

| | Example | |
|---|---|---|
| | (Compound | |
| Compound | No.) | Procedural notes |
| [chemical structure] | 1 (Cpd. 1) | Step S-5. 1-cycloheptylpiperazine (1.5 equiv.) was used. Step S-7. Same as above except 50 equiv of iodomethane was used. ¹H NMR (400 MHz, METHANOL-d₄) δ ppm 0.73-0.88 (m, 7 H) 0.93- 1.03 (m, 3 H) 1.05-1.14 (m, 8 H) 1.19 (s, 1 H) 1.25-1.31 (m, 2 H) 1.33-1.57 (m, 10 H) 1.59-1.65 (m, 3 H) 1.68-1.80 (m, 2 H) 2.34-2.54 (m, 6 H) 2.56 (dd, J = 8.28, 2.26 Hz, 1 H) 2.62 (td, J = 5.96, 2.38 Hz, 1 H) 3.11 (s, 2 H) 3.19-3.23 (m, 16 H) 3.31-3.59 (m, 4 H) 3.69 (br. s., 1 H) 4.74 (s, 10 H) 4.83 (d, J = 9.79 Hz, 1 H) 4.95 (d, J = 10.54 Hz, 1 H) 5.41- 5.67 (m, 3 H) 5.99 (d, J = 10.79 Hz, 1 H) 6.23 (dd, J = 14.93, 10.92 Hz, 1 H) |
| [chemical structure] | 2 (Cpd. 2) | Step S-5. 1-methylpiperazine (1.5 equiv.) was used. Step S-7. Same as above except 1.5 equiv of iodomethane was used. ¹H NMR (400 MHz, METHANOL-d₄) δ ppm 0.84-1.00 (m, 6 H) 1.03 (s, 1 H) 1.10 (d, J = 6.78 Hz, 2 H) 1.23 (s, 3 H) 1.31 (s, 5 H) 1.39 (d, J = 9.29 Hz, 2 H) 1.52 (d, J = 5.02 Hz, 2 H) 1.56- 1.71 (m, 2 H) 1.77 (s, 2 H) 1.95 (br. s., 1 H) 2.32 (s, 2 H) 2.43 (br. s., 2 H) 2.55 (d, J = 4.27 Hz, 2 H) 2.68 (dd, J = 8.03, 2.51 Hz, 1 H) 2.75 (d, J = 6.27 Hz, 1 H) 3.10-3.17 (m, 1 H) 3.31- 3.37 (m, 79 H) 3.43-3.72 (m, 6 H) 3.80 (br. s., 1 H) 4.86 (s, 41 H) 4.96 (d, J = 9.79 Hz, 1 H) 5.07 (d, J = 10.54 Hz, 1 H) 5.51 (s, 3 H) 5.54- 5.82 (m, 3 H) 6.11 (d, J = 10.79 Hz, 1 H) 6.28-6.40 (m, 1 H) |
| [chemical structure] | 3 (Cpd. 3) | Step S-5. 1-isopropylpiperazine (3.0 equiv.) was used. Step S-7. Same as above except 1.5 equiv of iodomethane was used. ¹H NMR (400 MHz, METHANOL-d₄) δ ppm 0.75-1.02 (m, 8 H) 1.03- 1.16 (m, 3 H) 1.17-1.28 (m, 3 H) 1.31 (br. s., 4 H) 1.35-1.57 (m, 7 H) 1.58-1.71 (m, 3 H) 1.77 (s, 2 H) 2.46-2.64 (m, 3 H) 2.67-2.69 (m, 16 H) 2.71-2.78 (m, 1 H) 2.94 (s, 1 H) 2.99-3.09 (m, 2 H) 3.15 (s, 1 H) 3.33 (dt, J = 3.26, 1.63 Hz, 42 H) 3.42-3.63 (m, 4 H) 3.77-3.94 (m, 4 H) 4.86 (s, 36 H) 4.96-5.04 (m, 1 H) 5.07 (d, J = 10.54 Hz, 1 H) 5.50-5.81 (m, 3 H) 6.12 (d, J = 10.04 Hz, 1 H) 6.35 (dd, J = 14.81, 10.79 Hz, 1 H) |
| [chemical structure] | 4 (Cpd. 4) | Step S-5. 3-Morpholinopropan-1- amine (3.0 equiv.) was used. Step S-7. Same as above except 50 equiv of iodomethane was used. ¹H NMR (400 MHz, METHANOL-d₄) δ ppm 0.72-0.87 (m, 7 H) 0.94 (d, J = 7.03 Hz, 1 H) 0.98 (d, J = 6.78 Hz, 2 H) 1.04-1.15 (m, 3 H) 1.15-1.30 (m, 9 H) 1.31-1.46 (m, 2 H) 1.46- 1.68 (m, 4 H) 1.92 (br. s., 1 H) 2.00- 2.19 (m, 1 H) 2.27-2.50 (m, 4 H) 2.50-2.71 (m, 1 H) 3.00-3.17 (m, 2 H) 3.21 (dt, J = 3.26, 1.63 Hz, 32 H) 3.35-3.57 (m, 2 H) 3.57-3.61 (m, 1 H) 3.69 (br. s., 1 H) 3.89 (br, s., 1 H) 4.46 (s, 1 H) 4.74 (s, 57 H) 4.95 (d, J = 10.54 Hz, 1 H) 5.10-5.32 (m, 1 H) 5.56 (dd, J = 14.93, 8.16 Hz, 2 H) 5.99 (d, J = 11.04 Hz, 1 H) 6.23 (dd, J = 14.81, 10.79 Hz, 1 H) |
| [chemical structure] | 5 (Cpd. 5) | Step S-5. N,N-dimethylpropane-1,3- diamine (3.0 equiv.) was used. Step S-7. Same as above except 200 equiv of iodomethane was used. ¹H NMR (400 MHz, METHANOL-d₄) δ ppm 0.74-0.88 (m, 9 H) 0.92- 1.02 (m, 3 H) 1.06-1.15 (m, 4 H) 1.15-1.30 (m, 9 H) 1.31-1.57 (m, 6 H) 1.61-1.69 (m, 2 H) 1.72-1.84 (m, 1 H) 1.85-1.99 (m, 2 H) 2.07- 2.13 (m, 1 H) 2.33-2.51 (m, 3 H) 2.51-2.70 (m, 2 H) 2.77 (s, 2 H) 2.99-3.07 (m, 5 H) 3.08-3.16 (m, 2 H) 3.21 (dt, J = 3.26, 1.63 Hz, 29 H) 3.24-3.31 (m, 1 H) 3.41 (td, J = 8.66, 4.77 Hz, 1 H) 3.59 (s, 1 H) 3.70 (br. s., 1 H) 4.33 (s, 1 H) 4.74 (s, 27 H) 4.81 (d, J = 9.79 Hz, 1 H) 4.95 (d, J = 10.54 Hz, 1 H) 5.39 (s, 1 H) 5.42- 5.69 (m, 3 H) 5.99 (d, J = 11.54 Hz, 1 H) 6.23 (dd, J = 14.68, 11.17 Hz, 1 H) |
| [chemical structure] | 6 (Cpd. 6) | Step S-5. 1-cyclohexylpiperazine (3.0 equiv.) was used. ¹H NMR (400 MHz, METHANOL-d₄) δ ppm 0.69-0.93 (m, 9 H) 0.99 (d, J = 6.78 Hz, 3 H) 1.08-1.22 (m, 7 H) 1.26-1.55 (m, 10 H) 1.60-1.66 (m, 3 H) 1.87-1.94 (m, 3 H) 2.15 (d, J = 10.79 Hz, 2 H) 2.32-2.51 (m, 3 H) 2.51-2.58 (m, 1 H) 2.63 (td, J = 5.96, 2.38 Hz, 1 H) 2.95 (s, 2 H) 3.19- 3.25 (m, 19 H) 3.36-3.55 (m, 5 H) 3.70 (d, J = 9.54 Hz, 1 H) 4.00 (q, J = 7.28 Hz, 2 H) 4.73 (s, 40 H) 4.88 (d, J = 9.79 Hz, 1 H) 4.95 (d, J = 10.54 Hz, 1 H) 5.44-5.71 (m, 3 H) 6.00 (d, J = 10.54 Hz, 1 H) 6.23 (dd, J = 15.06, 11.04 Hz, 1 H) |
| [chemical structure] | 7 (Cpd. 7) | Step 5-5, N,N-diethyl-N′- methylethane-1,2-diamine (3.0 equiv.) was used. ¹H NMR (400 MHz, METHANOL-d₄) δ ppm 0.71-0.91 (m, 9 H) 0.99 (d, J = 6.78 Hz, 3 H) 1.07-1.16 (m, 4 H) 1.19-1.46 (m, 12 H) 1.54 (dt, J = 13.93, 5.46 Hz, 2 H) 1.62-1.68 (m, 3 H) 2.31 (s, 1 H) 2.34-2.53 (m, 4 H) 2.56 (dd, J = 8.28, 2.26 Hz, 1 H) 2.60-2.65 (m, 1 H) 2.83 (s, 1 H) 2.89 (br. s., 1 H) 2.96 (br. s., 4 H) 3.11 (s, 1 H) 3.21 (dt, J = 3.26, 1.63 Hz, 10 H) 3.34 (q, J = 7.19 Hz, 4 H) 3.38-3.44 (m, 1 H) 3.50-3.64 (m, 1 H) 3.69 (br. s., 1 H) 4.74 (s, 17 H) 4.79-4.98 (m, 2 H) 5.39 (s, 1 H) 5.45-5.70 (m, 3 H) 6.00 (d, J = 10.79 Hz, 1 H) 6.23 (dd, J = 14.81, 10.54 Hz, 1H) |
| [chemical structure] | 8 (Cpd. 8) | Step S-5. 1-Methyl-1,4-diazepane (3.0 equiv.) was used. ¹H NMR (400 MHz, METHANOL-d₄) δ ppm 0.65 (br. s., 1 H) 0.69-0.92 (m, 8 H) 0.99 (d, J = 6.78 Hz, 2 H) 1.06-1.27 (m, 9 H) 1.32-1.43 (m, 2 H) 1.46 (br. s., 1 H) 1.53 (dt, J = 13.87, 5.62 Hz, 2 H) 1.59 (br. s., 1 H) 1.62-1.68 (m, 2 H) 2.11 (br. s., 2 H) 2.32-2.58 (m, 4 H) 2.63 (td, J = 5.90, 2.26 Hz, 1 H) 3.11 (s, 5 H) 3.19-3.23 (m, 20 H) 3.37-3.64 (m, 5 H) 3.65-3.79 (m, 2 H) 3.85 (br. s., 1 H) 4.74 (s, 25 H) 4.79 (s, 1 H) 4.88 (d, J = 9.79 Hz, 1 H) 4.95 (d, J = 10.79 Hz, 1 H) 5.46-5.68 (m, 2 H) 6.00 (d, J = 10.29 Hz, 1 H) 6.15-6.35 (m, 1 H) |
| [chemical structure] | 9 (Cpd. 9) | Step 5-5. 2-Methyl-2,6- diazaspiro[3.3]heptane (5.0 equiv.) was used. ¹H NMR (400 MHz, METHANOL-d₄) δ ppm 0.73-0.98 (m, 7 H) 0.99- 1.11 (m, 3 H) 1.14 (br. s., 1 H) 1.16- 1.28 (m, 6 H) 1.36 (s, 2 H) 1.45 (d, J = 11.54 Hz, 3 H) 1.55-1.77 (m, 3 H) 1.79-2.00 (m, 3 H) 2.01-2.22 (m, 2 H) 2.28 (br, s., 2 H) 2.81 (s, 1 H) 3.03 (s, 1 H) 3.11 (s, 1 H) 3.21 (dt, J = 3.26, 1.63 Hz, 44 H) 3.32-3.59 (m, 1 H) 3.66-3.91 (m, 2 H) 4.74 (s, 51 H) 5.20-5.29 (m, 1 H) 5.37-5.58 (m, 2 H) 5.82 (d, J = 15.06 Hz, 1 H) 6.11- 6.24 (m, 1 H) |
| [chemical structure] | 10 (Cpd. 10) | Step S-5. 1-Isopropyl-1,4-diazepane dihydrochloride (3.0 equiv.) was used. Step S-7. Same as above except 1000 equiv of iodomethane was used. ¹H NMR (400 MHz, METHANOL-d₄) δ ppm 0.71-0.88 (m, 10 H) 0.95- 1.01 (m, 2 H) 1.03 (d, J = 6.27 Hz, 1 H) 1.10-1.16 (m, 3 H) 1.16-1.24 (m, 8 H) 1.25-1.31 (m, 3 H) 1.31- 1.37 (m, 4 H) 1.37-1.42 (m, 2 H) 1.49-1.57 (m, 3 H) 1.60-1.70 (m, 3 H) 1.99-2.16 (m, 2 H) 2.31-2.52 (m, 3 H) 2.56 (dd, J = 8.16, 2.38 Hz, 1 H) 2.63 (td, J = 5.77, 2.26 Hz, 1 H) 2.90 (s, 2 H) 3.18-3.24 (m, 29 H) 3.31 (d, J = 5.52 Hz, 1 H) 3.36-3.58 (m, 4 H) 3.63-3.92 (m, 4 H) 4.00 (s, 1 H) 4.72-4.76 (m, 27 H) 4.89 (d, J = 9.54 Hz, 1 H) 4.95 (d, J = 10.54 Hz, 1 H) 5.43-5.74 (m, 3 H) 6.00 (d, J = 10.79 Hz, 1 H) 6.23 (dd, J = 14.68, 10.67 Hz, 1 H) |
| [chemical structure] | 11 (Cpd. 11) | Step S-5. 1-Cyclohexyl-1,4- diazepane (3.0 equiv.) was used. ¹H NMR (400 MHz, METHANOL-d₄) δ ppm 0.70-0.89 (m, 10 H) 0.99 (d, J = 6.78 Hz, 3 H) 1.03 (d, J = 6.53 Hz, 1 H) 1.06-1.17 (m, 5 H) 1.17-1.24 (m, 3 H) 1.27-1.42 (m, 6 H) 1.44- 1.57 (m, 5 H) 1.59-1.70 (m, 4 H) 1.85-1.96 (m, 2 H) 2.00-2.21 (m, 4 H) 2.31-2.53 (m, 4 H) 2.56 (dd, J = 8.28, 2.26 Hz, 1 H) 2.63 (td, J = 5.90, 2.26 Hz, 1 H) 2.83-2.98 (m, 3 H) 3.18-3.24 (m, 9 H) 3.30-3.46 (m, 4 H) 3.51-3.75 (m, 5 H) 3.79 (br, s., 1 H) 3.84 (br. s., 1 H) 4.74 (s, 9 H) 4.89 (d, J = 9.54 Hz, 1 H) 4.95 (d, J = 10.79 Hz, 1 H) 5.46-5.70 (m, 3 H) 6.00 (d, J = 10.54 Hz, 1 H) 6.23 (dd, J = 15.06, 10.79 Hz, 1 H) |
| [chemical structure] | 12 (Cpd. 12) | Step S-5. 2-Cycloheptyl-2,6- diazaspiro[3.3]heptane (3.0 equiv.) was used. ¹H NMR (400 MHz, METHANOL-d₄) δ ppm 0.74-0.89 (m, 10 H) 0.98 (d, J = 6.78 Hz, 3 H) 1.11 (s, 5 H) 1.19 (s, 1 H) 1.23-1.30 (m, 1 H) 1.31-1.59 (m, 16 H) 1.64 (d, J = 1.00 Hz, 3 H) 1.70-1.86 (m, 1 H) 2.42 (d, J = 3.76 Hz, 4 H) 2.56 (dd, J = 8.03, 2.26 Hz, 1 H) 2.60-2.66 (m, 1 H) 2.88 (s, 3 H) 3.37-3.46 (m, 1 H) 3.50 (s, 1 H) 3.68 (br, s., 1 H) 4.01-4.16 (m, 1 H) 4.26 (br, s., 4 H) 4.49 (s, 2 H) 4.79 (d, J = 9.79 Hz, 1 H) 4.94 (d, J = 10.54 Hz, 1 H) 5.41-5.71 (m, 3 H) 5.99 (d, J = 10.54 Hz, 1 H) 6.19 (s, 1 H) |
| [chemical structure] | 13 (Cpd. 13) | Step S-5. 4-(Pyrrolidin-1- yl)piperidine (1.5 equiv.) was used. ¹H NMR (400 MHz, METHANOL-d4) δ ppm 0.71-0.94 (m, 7 H) 0.98 (d, J = 6.78 Hz, 2 H) 1.26-1.31 (m, 1 H) 1.31-1.59 (m, 6 H) 1.34-1.44 (m, 3 H) 1.76-1.91 (m, 3 H) 1.97 (d, J = 12.55 Hz, 2 H) 2.43 (d, 1 = 3.76 Hz, 2 H) 2.53-2.68 (m, 2 H) 2.69-2.83 (m, 2 H) 2.85-2.98 (m, 3 H) 3.34- 3.48 (m, 1 H) 3.70 (br. s., 1 H) 4.47 (s, 1 H) 4.79-4.88 (m, 1 H) 4.95 (d, J = 10.54 Hz, 1 H) 5.42-5.57 (m, 1 H) 5.57-5.69 (m, 1 H) 6.00 (d, J = 10.79 Hz, 1 H) 6.23 (dd, J = 15.18, 10.67 Hz, 1 H) |
| [chemical structure] | 14 (Cpd. 14) | Step S-5. 1,4′-Bipiperidine (1.5 equiv.) was used. ¹H NMR (400 MHz, CHLOROFORM- d) δ ppm 0.78-0.93 (m, 9 H) 1.01 (d, J = 6.78 Hz, 3 H) 1.15-1.22 (m, 4 H) 1.25 (d, J = 10.79 Hz, 2 H) 1.29- 1.37 (m, 2 H) 1.40-1.49 (m, 4 H) 1.49-1.58 (m, 2 H) 1.79-2.01 (m, 6 H) 2.27 (br. s., 1 H) 2.33-2.44 (m, 2 H) 2.43-2.57 (m, 3 H) 2.60 (dd, J = 7.53, 2.26 Hz, 1 H) 2.70 (td, J = 5.90, 2.26 Hz, 1 H) 3.29-3.53 (m, 2 H) 3.52-3.80 (m, 5 H) 4.33 (d, J = 6.78 Hz, 2 H) 4.50 (t, J = 11.92 Hz, 1 H) 4.92 (d, J = 9.54 Hz, 1 H) 5.08 (d, J = 10.54 Hz, 1 H) 5.41-5.74 (m, 3 H) 6.00 (d, J = 10.79 Hz, 1 H) 6.18 (dd, J = 14.81, 10.79 Hz, 1 H) |
| [chemical structure] | 15 (Cpd. 15) | Step S-5. N,N′-dimethylpiperidin-4- amine (1.5 equiv.) was used. |
| [chemical structure] | 16 (Cpd. 16) | Step S-5. (1R,4R)-2-methyl-2,5- diazabicyclo[2.2.1]heptane (1.0 equiv.) was used. ¹H NMR (400 MHz, METHANOL-d₄) δ ppm 0.69-0.88 (m, 11 H) 0.99 (d, J = 6.78 Hz, 3 H) 1.03 (d, 3 = 6.27 Hz, 1 H) 1.08-1.17 (m, 5 H) 1.17-1.24 (m, 4 H) 1.24-1.32 (m, 2 H) 1.32- 1.47 (m, 4 H) 1.47-1.61 (m, 3 H) 1.62-1.68 (m, 3 H) 2.22-2.31 (m, 1 H) 2.34-2.54 (m, 5 H) 2.56 (dd, J = 8.03, 2.26 Hz, 1 H) 2.63 (td, J = 5.90, 2.26 Hz, 1 H) 3.14 (br. s., 3 H) 3.19-3.24 (m, 16 H) 3.25 (s, 2 H) 3.39-3.45 (m, 1 H) 3.48-3.66 (m, 3 H) 3.66-3.78 (m, 1 H) 3.86 (d, J = 12.80 Hz, 1 H) 3.93-4.06 (m, 1 H) 4.39 (br. s., 1 H) 4.74 (s, 27 H) 4.86 (d, J = 9.54 Hz, 1 H) 4.95 (d, J = 10.79 Hz, 1 H) 5.47-5.70 (m, 3 H) 6.00 (d, J = 11.04 Hz, 1 H) 6.23 (dd, J = 14.56, 10.54 Hz, 1 H) |

### C7 Quaternary Amine Analogs Originating from E7107

[chemical structure]

Step S-8: A solution of E7107 (I, 1.0 equiv.) in dichloromethane (0.01M) under nitrogen at room temperature was treated with alkyl halide (see Table 2). The reaction was stirred for 4 hours, or until the reaction was determined to be complete by LCMS or TLC. The reaction was concentrated in vacuo and was purified by silica gel column chromatography (dichloromethane/methanol as eluant) to afford the desired product J (compounds 17-20).

Examples 17-20 were prepared using the above scheme.

TABLE 2

| | Example | |
|---|---|---|
| | (Compound | |
| Compound | No.) | Procedural notes |
| [chemical structure] | 17 (Cpd. 17) | Step S-8. Methyl iodide (10 equiv.) was used. ¹H NMR (400 MHz, METHANOL-d₄) δ ppm 0.87-0.99 (m, 10 H) 1.19- 1.34 (m, 5 H) 1.35-1.42 (m, 5 H) 1.47-1.71 (m, 12 H) 1.78-1.91 (m, 8H) 2.23 (d, J = 10.92 Hz, 2 H) 2.52- 2.66 (m, 3 H) 2.69 (dd, J = 7.91, 2.26 Hz, 1 H) 2.92 (td, J = 5.80, 2.32 Hz, 1 H) 3.05 (s, 3 H) 3.49-3.67 (m, 6 H) 3.67-3.85 (m, 4 H) 5.00 (d, J = 9.66 Hz, 1 H) 5.08 (d, J = 10.67 Hz, 1 H) 5.62 (dd, J = 15.18, 9.79 Hz, 1 H) 5.71-5.79 (m, 1 H) 5.89 (d, J = 15.18 Hz, 1H) 6.16 (d, J = 10.92 Hz, 1 H) 6.55 (dd, J = 15.25, 10.98 Hz, 1 H) |
| [chemical structure] | 18 (Cpd. 18) | Step S-8. Benzyl bromide (10 equiv.) was used. ¹H NMR (400 MHz, METHANOL-d₄) δ ppm 0.86 (br. s., 3 H) 0.89-0.99 (m, 7 H) 1.12 (br. s., 2 H) 1.22-1.41 (m, 8 H) 1.45-1.62 (m, 4 H) 1.62- 1.73 (m, 8 H) 1.79 (s, 3 H) 1.88 (dd, J = 14.05, 5.40 Hz, 3 H) 1.95-2.09 (m, 2 H) 2.24-2.33 (m, 2 H) 2.53 (d, J = 3.64 Hz, 3 H) 2.69 (dd, J = 7.91, 2.13 Hz, 1 H) 2.91 (td, J = 5.83, 2.13 Hz, 1 H) 3.13-3.26 (m, 2 H) 3.50- 3.64 (m, 1 H) 3.64-3.90 (m, 5 H) 4.18 (br. s., 3 H) 4.60 (br, s., 2 H) 4.85 (s, 10 H) 4.95 (d, J = 5.90 Hz, 1 H) 5.06 (d, J = 10.54 Hz, 1 H) 5.53- 5.64 (m, 1 H) 5.68 (br. s., 1 H) 5.89 (d, J = 15.18 Hz, 1 H) 6.14 (d, J = 10.67 Hz, 1H) 6.54 (dd, J = 15.25, 10.98 Hz, 1 H) 7.52-7.62 (m, 5 H) |
| [chemical structure] | 19 (Cpd. 19) | Step S-8. Allyl bromide (10 equiv.) was used. ¹H NMR (400 MHz, METHANOL-d₄) δ ppm 0.82-1.03 (m, 26 H) 1.19- 1.34 (m, 16 H) 1.34-1.42 (m, 13 H) 1.47-1.71 (m, 29 H) 1.77-1.83 (m, 8 H) 1.89 (dd, J = 13.99, 5.33 Hz, 14 H) 2.25 (br. s., 5 H) 2.51-2.63 (m, 7 H) 2.69 (dd, J = 7.97, 2.32 Hz, 2 H) 2.92 (td, J = 5.83, 2.38 Hz, 2 H) 3.46- 3.61 (m, 8 H) 3.64 (br. s., 5 H) 3.76- 3.94 (m, 10 H) 4.18 (d, J = 7.15 Hz, 6 H) 5.00 (d, J = 9.66 Hz, 2 H) 5.08 (d, J = 10.67 Hz, 2 H) 5.62 (dd, J = 15.18, 9.79 Hz, 2 H) 5.71-5.79 (m, 8 H) 5.89 (d, J = 15.18 Hz, 2 H) 6.04-6.18 (m, 5 H) 6.55 (dd, J = 15.25, 10.98 Hz, 2 H) |
| [chemical structure] | 20 (Cpd. 20) | Step S-8. Allyl iodide (10 equiv.) was used. ¹H NMR (400 MHz, METHANOL-d₄) δ ppm 0.85-1.01 (m, 11 H) 1.19- 1.34 (m, 6 H) 1.34-1.43 (m, 6 H) 1.47-1.71 (m, 11 H) 1.80 (s, 4 H) 1.89 (dd, J = 14.05, 5.40 Hz, 5 H) 2.25 (br. s., 2 H) 2.51-2.71 (m, 5 H) 2.92 (td, J = 5.80, 2.20 Hz, 1 H) 3.45-3.61 (m, 4 H) 3.65 (br. s., 2 H) 3.75- 4.01 (m, 5 H) 4.01-4.21 (m, 3 H) 5.00 (d, J = 9.66 Hz, 1 H) 5.08 (d, J = 10.67 Hz, 1 H) 5.58-5.67 (m, 1 H) 5.71-5.83 (m, 4 H) 5.89 (d, J = 15.31 Hz, 1 H) 6.05-6.19 (m, 2 H) 6.55 (dd, J = 15.31, 10.92 Hz, 1 H) |

### C6 Quaternary Amine Analogs Originating from Pladienolide B

[chemical structure]

[chemical structure]

Step S-9: Same as Step S-1 (A to B).

Step S-10: A solution of alcohol B (1.0 equiv.) in dichloromethane (0.09M) under nitrogen at 0° C. was treated with sodium hydride (3.0 equiv., 60% dispersion in mineral oil) and methyl iodide (20.0 equiv.). The reaction was stirred for two days, or until the reaction was determined to be complete by LCMS or TLC. The reaction was quenched with cold saturated sodium bicarbonate solution and diluted with dichloromethane. The organic layer was separated, dried over magnesium sulfate, filtered, and concentrated in vacuo. The resulting oil was purified by silica gel column chromatography (dichloromethane/methanol as eluant) to afford the desired product (K).

Step S-11: A solution of methyl ether K (1.0 equiv.) in MeOH (0.2M) under nitrogen at 0° C. was treated with guanidine nitrate (1.1 equiv.) dropwise over three minutes. The reaction was allowed to warm to room temperature and stirred for 16 hours, or until the reaction was determined to be complete by LCMS or TLC. The reaction was diluted with ethyl acetate and saturated ammonium chloride solution and the organic layer was washed with brine, dried over magnesium sulfate, filtered, and concentrated in vacuo. The resulting oil was purified by silica gel column chromatography (hexanes/ethyl acetate as eluant) to afford the desired product (L).

Step S-12: A solution of alcohol L (1.0 equiv.) in dichloromethane (0.05M) under nitrogen at room temperature was treated with DMAP (5.0 equiv.), triethylamine (5.0 equiv.), and 4-nitrophenylchloroformate (3.0 equiv.). The reaction was stirred for 16 hours, or until the reaction was determined to be complete by LCMS or TLC. The reaction was diluted with dichloromethane and washed with brine, dried over magnesium sulfate, filtered, and concentrated in vacuo. The resulting oil was purified by silica gel column chromatography (hexanes/ethyl acetate as eluant) to afford the desired product (M).

Step S-13: A solution of carbonate M (1.0 equiv.) in methyl tert-butyl ether (0.01M) under nitrogen at room temperature was treated with N,N-diisopropylethylamine (10.0 equiv.) and amine (see Table 3). The reaction was stirred for 16 hours, or until the reaction was determined to be complete by LCMS or TLC. The reaction was diluted with methyl tert-butyl ether and the organic layer was washed with saturated ammonium bicarbonate solution, brine, dried over magnesium sulfate, filtered, and concentrated in vacuo. The resulting oil was purified by silica gel column chromatography (hexanes/ethyl acetate as eluant) to afford the desired product (N).

Step S-14: A solution of silyl ether N (1.0 equiv.) in dichloromethane (0.01M) under nitrogen was cooled to −78° C. and was treated with N,N-diisopropylethylamine (286 equiv.) followed by HF pyridine (39 equiv.). The reaction was allowed to warm to room temperature over 18 hours, or until the reaction was determined to be complete by LCMS or TLC. The reaction was then diluted with dichloromethane, the organic layer separated, dried over magnesium sulfate, filtered, and concentrated in vacuo. The resulting oil was purified by silica gel column chromatography (dichloromethane/methanol as eluant) to afford the desired product (O).

Step S-15: A solution of carbamate O (1.0 equiv.) in dichloromethane (0.01M) under nitrogen at room temperature was treated with methyl iodide (139 equiv.) The reaction was stirred for 64 hours, or until the reaction was determined to be complete by LCMS or TLC. The reaction was concentrated in vacuo and was purified by silica gel column chromatography (dichloromethane/methanol as eluant) to afford the desired product P (compounds 21 and 22).

Examples 21 and 22 were prepared using the above scheme.

TABLE 3

| | Example | |
|---|---|---|
| | (Compound | |
| Compound | No.) | Procedural notes |
| [chemical structure] | 21 (Cpd. 21) | Step S-13. 1-Cycloheptylpiperazine (1.5 equiv.) was used. ¹H NMR (400 MHz, METHANOL-d₄) δ ppm 0.81-1.03 (m, 11 H) 1.10 (d, J = 6.78 Hz, 3 H) 1.18-1.35 (m, 5 H) 1.40-1.61 (m, 7 H) 1.61-1.70 (m, 8 H) 1.75-1.90 (m, 7 H) 2.18 (s, 2 H) 2.22 (d, J = 9.79 Hz, 1 H) 2.44-2.63 (m, 4 H) 2.68 (dd, J = 8.16, 2.26 Hz, 1 H) 2.75 (td, J = 5.90, 2.26 Hz, 1H) 3.03 (s, 3 H) 3.34-3.34 (m, 3 H) 3.49-3.70 (m, 6 H) 3.73 (br. s., 2 H) 3.78-3.88 (m, 1 H) 4.04 (br. s., 2 H) 5.07 (dd, J = 10.16, 1.88 Hz, 2 H) 5.57-5.69 (m, 2 H) 5.69- 5.79 (m, 2 H) 6.11 (d, J = 10.92 Hz, 1 H) 6.35 (dd, J = 15.00, 10.73 Hz, 1 H) |
| [chemical structure] | 22 (Cpd. 22) | Step S-13. 1-Cyclooctylpiperazine (1.5 equiv.) was used. Step S-14. HF pyridine (76 equiv.) and N,N′-diisopropylethylamine (393 equiv.) was used. ¹H NMR (400 MHz, METHANOL-d₄) δ ppm 0.82-1.03 (m, 11 H) 1.10 (d, J = 6.65 Hz, 3 H) 1.18-1.36 (m, 7 H) 1.45-1.73 (m, 17 H) 1.75- 1.78 (m, 3 H) 1.80-1.94 (m, 4 H) 2.10- 2.20 (m, 3 H) 2.42-2.62 (m, 4 H) 2.68 (dd, J = 8.22, 2.32 Hz, 1 H) 2.75 (td, J = 5.93, 2.20 Hz, 1 H) 3.01 (s, 3 H) 3.34 (s, 3 H) 3.43-3.60 (m, 4 H) 3.66 (d, J = 11.42 Hz, 4 H) 3.76 (d, J = 6.65 Hz, 1 H) 3.80-3.88 (m, 1 H) 4.15 (br. s., 2 H) 5.07 (dd, J = 10.23, 1.94 Hz, 2 H) 5.57-5.69 (m, 2 H) 5.69-5.79 (m, 2 H) 6.11 (d, J = 10.79 Hz, 1 H) 6.35 (dd, J = 14.74, 10.60 Hz, 1 H) |

### C6-OMe Analogs Originating from E7107

[chemical structure]

[chemical structure]

Example 23 was made by the following scheme:

Step S-16: A solution of E7107 (I, 1.0 equiv.) under nitrogen in dichloromethane (0.1M) was treated with DMAP (10.0 equiv.) and N,N-diisopropylethylamine (10.0 equiv.). The reaction was cooled to 0° C. and TESCl (10.0 equiv.) was added. The reaction was allowed to warm to room temperature and stirred for two days, or until the reaction was determined to be complete by LCMS or TLC. The reaction was quenched with saturated sodium bicarbonate solution, the organic layer was separated and washed with brine, dried over magnesium sulfate, filtered, and concentrated in vacuo. The resulting oil was purified by silica gel column chromatography (hexanes/ethyl acetate as eluant) to afford the desired product (O).

Step S-17: A solution of alcohol Q (1.0 equiv.) in dichloromethane (0.09M) under nitrogen at 0° C. was treated with sodium hydride (3.0 equiv., 60% dispersion in mineral oil) and methyl iodide (20 equiv.). The reaction was stirred for two days, or until the reaction was determined to be complete by LCMS or TLC. The reaction was quenched with cold saturated sodium bicarbonate solution in dichloromethane. The organic layer was separated, dried over magnesium sulfate, filtered, and concentrated in vacuo. The resulting oil was purified by silica gel column chromatography (dichloromethane/methanol as eluant) to afford the desired product (R).

Step S-18: A solution of methyl ether R (1.0 equiv.) in dichloromethane (0.01M) under nitrogen was cooled to −78° C. and was treated with N,N-diisopropylethylamine (120 equiv.) followed by HF pyridine (17 equiv.). The reaction was allowed to warm to room temperature over 18 hours, or until the reaction was determined to be complete by LCMS or TLC. The reaction was diluted with dichloromethane, the organic layer was separated, dried over magnesium sulfate, filtered, and concentrated in vacuo. The resulting oil was purified by HPLC (acetonitrile/0.1% aqueous formic acid as eluant) to afford the desired product (S; compound 23). ¹H NMR (400 MHz, METHANOL-d₄) δ ppm 0.00 (s, 1H) 0.74-0.87 (m, 10H) 1.08-1.22 (m, 5H) 1.25 (s, 3H) 1.30-1.49 (m, 6H) 1.49-1.59 (m, 8H) 1.66-1.71 (m, 4H) 1.71-1.79 (m, 5H) 2.09-2.15 (m, 2H) 2.37-2.51 (m, 3H) 2.57 (dd, J=7.91, 2.26 Hz, 1H) 2.80 (td, J=5.87, 2.20 Hz, 1H) 2.91 (s, 3H) 3.22 (s, 3H) 3.36-3.52 (m, 6H) 3.55 (br. s., 1H) 3.60 (br. s., 2H) 3.66-3.76 (m, 1H) 3.91 (br. s., 2H) 4.96 (dd, J=10.16, 3.51 Hz, 2H) 5.49 (dd, J=15.25, 9.85 Hz, 1H) 5.64 (dd, J=15.25, 9.60 Hz, 1H) 5.77 (d, J=15.18 Hz, 1H) 6.04 (d, J=10.92 Hz, 1H) 6.43 (dd, J=15.31, 10.92 Hz, 1H)

[chemical structure]

[chemical structure]

Example 24 was made by the following scheme:

Step S-19: Same as step S-16 (I to Q).

Step S-20: A solution of alcohol Q (1.0 equiv.) in dichloromethane (0.02M) under nitrogen was cooled to −78° C. and treated with DMAP (10 equiv.), diisopropylethylamine (240 equiv.), and trifluoroacetic anhydride. The reaction was allowed to warm to room temperature over three hours, or until the reaction was determined to be complete by LCMS or TLC. The reaction was quenched with cold saturated sodium bicarbonate solution in dichloromethane. The organic layer was separated, dried over magnesium sulfate, filtered, and concentrated in vacuo. The resulting oil was purified by silica gel column chromatography (hexane/ethyl acetate as eluant) to afford the desired product (T).

Step S-21: A solution of trifluoroacetate T (1.0 equiv.) in dichloromethane (0.01M) under nitrogen was cooled to −78° C. and was treated with N,N-diisopropylethylamine (287 equiv.) followed by HF pyridine (192 equiv.). The reaction was allowed to warm to 0° C. over six hours, or until the reaction was determined to be complete by LCMS or TLC. The reaction was quenched with saturated sodium bicarbonate solution and diluted with dichloromethane, the organic layer was separated, dried over magnesium sulfate, filtered, and concentrated in vacuo. The resulting oil was purified by silica gel column chromatography (dichloromethane/methanol as eluant) to afford the desired product (U).

Step S-22: A solution of trifluoroacetate U (1.0 equiv.) in dichloromethane (0.09M) under nitrogen at room temperature was treated with methyl iodide (10 equiv.). The reaction was stirred for two days, or until the reaction was determined to be complete by LCMS or TLC. The reaction was concentrated in vacuo and purified by HPLC (acetonitrile/0.1% aqueous formic acid as eluant) to afford the desired product (V; compound 24). ¹H NMR (400 MHz, METHANOL-d₄) δ ppm 0.74-0.88 (m, 11H) 1.12-1.22 (m, 7H) 1.25 (s, 4H) 1.30-1.48 (m, 4H) 1.48-1.64 (m, 12H) 1.66-1.80 (m, 10H) 2.06-2.26 (m, 3H) 2.42-2.59 (m, 7H) 2.77-2.82 (m, 1H) 2.91 (s, 3H) 3.36-3.56 (m, 6H) 3.59 (br. s., 2H) 3.75 (dd, J=9.66, 3.89 Hz, 1H) 3.91 (d, J=15.43 Hz, 2H) 4.97 (d, J=10.67 Hz, 1H) 5.10 (d, J=8.53 Hz, 1H) 5.55-5.68 (m, 2H) 5.78 (d, J=15.18 Hz, 1H) 6.05 (d, J=11.04 Hz, 1H) 6.43 (dd, J=15.25, 10.98 Hz, 1H)

### C21-Ketone Analog from E7107

[chemical structure]

Example 25 was made by the following scheme:

Step S-23: A solution of E7107 (I, 1.0 equiv.) in 1,2-dichloroethane (0.1M) under nitrogen at 0° C. was treated Dess-Martin periodinane (1.0 equiv.). The reaction was allowed to warm to room temperature and stirred for 3 hours, or until the reaction was determined to be complete by LCMS or TLC. The reaction was concentrated in vacuo and was purified by HPLC (acetonitrile/0.1% aqueous formic acid as eluant) to afford the desired product (W).

Step S-24: A solution of ketone W (1.0 equiv.) in dichloromethane (0.01M) under nitrogen at room temperature was treated with methyl iodide (85 equiv.) The reaction was stirred for 10 days, or until the reaction was determined to be complete by LCMS or TLC. The reaction was concentrated in vacuo and was purified by silica gel column chromatography (dichloromethane/methanol as eluant) to afford the desired product (X; compound 25). ¹H NMR (400 MHz, METHANOL-d₄) ppm 0.87-1.05 (m, 9H) 1.09 (d, J=7.15 Hz, 3H) 1.24-1.29 (m, 4H) 1.31 (s, 3H) 1.36 (s, 3H) 1.41 (d, J=8.78 Hz, 2H) 1.57-1.69 (m, 9H) 1.80 (s, 3H) 1.86-1.94 (m, 9H) 2.23 (br. s., 2H) 2.54-2.62 (m, 4H) 2.78 (dd, J=8.28, 2.13 Hz, 1H) 2.92-2.96 (m, 1H) 3.14-3.16 (m, 1H) 3.49-3.54 (m, 2H) 3.58 (br. s., 2H) 3.72 (br. s., 2H) 3.81 (s, 1H) 4.98-5.04 (m, 2H) 5.08 (d, J=10.67 Hz, 1H) 5.63 (dd, J=14.90, 12.40 Hz, 1H) 5.75 (dd, J=15.60, 10.20 Hz, 1H) 5.88 (d, J=15.31 Hz, 1H) 6.16 (d, J=10.92 Hz, 1H) 6.55 (dd, J=15.25, 10.98 Hz, 1H)

### C6 Quaternary Amine Salt Analogs

[chemical structure]

[chemical structure]

Examples 26-28 (Table 4) were prepared as follows:

Step S-25: Same as Step S-1 (A to B).

Step S-26: A solution of alcohol B (1.0 equiv.) in 1,2-dichloroethane (0.05M) under nitrogen at 0° C. was treated with DMAP (3.0 equiv.), N,N-diisopropylethylamine (20.0 equiv.), and 4-nitrophenylchloroformate (10.0 equiv.). The reaction was stirred for 16 hours, or until the reaction was determined to be complete by LCMS or TLC. The reaction was diluted with dichloromethane and washed with brine, dried over magnesium sulfate, filtered, and concentrated in vacuo. The resulting oil was purified by silica gel column chromatography (hexanes/ethyl acetate as eluant) to afford the desired product (Y).

Step S-27: A solution of carbonate Y (1.0 equiv.) in dichloromethane (0.04M) under nitrogen at room temperature was treated with N,N-diisopropylethylamine (15.0 equiv) and amine (see Table 4). The reaction was stirred for 16 hours, or until the reaction was determined to be complete by LCMS or TLC. The reaction was diluted with dichloromethane and washed with saturated sodium bicarbonate solution, brine, dried over magnesium sulfate, filtered, and concentrated in vacuo. The resulting oil was purified by silica gel column chromatography (hexanes/ethyl acetate as eluant) to afford the desired product (Z).

Step S-28: A solution of silyl ether Z (1.0 equiv.) in dichloromethane (0.01M) under nitrogen was cooled to −78° C. and was treated with N,N-diisopropylethylamine (286 equiv.) followed by HF pyridine (39 equiv.). The reaction was allowed to warm to room temperature over 18 hours, or until the reaction was determined to be complete by LCMS or TLC. The reaction was diluted with dichloromethane and saturated ammonium chloride solution, the organic layer was separated, dried over magnesium sulfate, filtered, and concentrated in vacuo. The resulting oil was purified by silica gel column chromatography (hexanes/ethyl acetate as eluant) to afford the desired product (AA).

Step S-29: A solution of carbamate AA (1.0 equiv.) in dichloromethane (0.09M) under nitrogen at room temperature was treated with methyl iodide (50 equiv.). The reaction was stirred for two weeks, or until the reaction was determined to be complete by LCMS or TLC. The reaction was concentrated in vacuo and was purified by silica gel column chromatography (dichloromethane/methanol as eluant) to afford the desired products BB (Compounds 26-28).

TABLE 4

| | Example | |
|---|---|---|
| | (Compound | |
| Compound | No.) | Procedural notes |
| [chemical structure] | 26 (Cpd. 26) | Step S-27: 1- Cycloheptylpiperazine (1.0 equiv.) was used. ¹H NMR (400 MHz, METHANOL-d₄) ppm 0.87-0.99 (m, 9 H) 1.11 (d, J = 6.78 Hz, 3 H) 1.22 (td, J = 7.40, 4.52 Hz, 1 H) 1.44-1.70 (m, 16 H) 1.77 (s, 3 H) 1.81-1.92 (m, 4 H) 2.08 (s, 3 H) 2.20-2.30 (m, 2 H) 2.30-2.45 (m, 1 H) 2.46-2.55 (m, 3 H) 2.58- 2.72 (m, 2 H) 2.75 (td, J = 5.90, 2.01 Hz, 1 H) 3.05 (s, 3 H) 3.37 (s, 2 H) 3.51-3.60 (m, 3 H) 3.60- 3.71 (m, 3 H) 3.73-3.87 (m, 3 H) 4.04 (br. s., 2 H) 5.00-5.16 (m, 2 H) 5.63-5.83 (m, 3 H) 6.12 (d, J = 10.79 Hz, 1 H) 6.35 (dd, J = 15.06, 10.79 Hz, 1 H) |
| [chemical structure] | 27 (Cpd. 27) | Step S-27: 1- Isopropylpiperazine (1.0 equiv.) was used. Step S-29: Methyl iodide (92 equiv.) was used. ¹H NMR (400 MHz, METHANOL-d₄) ppm 0.85-0.94 (m, 7 H) 0.96 (t, J = 7.40 Hz, 3 H) 1.10 (d, J = 6.78 Hz, 3 H) 1.22 (td, J = 7.50, 4.33 Hz, 1 H) 1.31 (br. s., 1 H) 1.42-1.58 (m, 10 H) 1.58- 1.69 (m, 5 H) 1.77 (s, 3 H) 2.07 (s, 3 H) 2.37 (br. s., 1 H) 2.46-2.55 (m, 3 H) 2.59-2.70 (m, 2 H) 2.72- 2.77 (m, 1 H) 3.02-3.06 (m, 3 H) 3.46-3.62 (m, 5 H) 3.70 (br. s., 1 H) 3.83 (dd, J = 9.91, 3.76 Hz, 1 H) 3.88-3.95 (m, 1 H) 4.06 (d, J = 12.67 Hz, 2 H) 5.08 (dd, J = 10.10, 3.70 Hz, 2 H) 5.65-5.74 (m, 2 H) 5.79 (dd, J = 15.06, 9.66 Hz, 1 H) 6.12 (d, J = 10.79 Hz, 1 H) 6.35 (dd, J = 14.62, 10.73 Hz, 1 H) |
| [chemical structure] | 28 (Cpd. 28) | Step S-27: N-Methylpiperazine (1.0 equiv.) was used. ¹H NMR (400 MHz, METHANOL-d₄) ppm 0.86-1.01 (m, 10 H) 1.07-1.12 (m, 3 H) 1.22 (td, J = 7.56, 4.45 Hz, 1 H) 1.45- 1.69 (m, 10 H) 1.75-1.80 (m, 3 H) 2.07 (s, 3 H) 2.33-2.44 (m, 1 H) 2.46-2.55 (m, 3 H) 2.58-2.70 (m, 2 H) 2.75 (td, J = 5.90, 2.26 Hz, 1 H) 3.27 (s, 6 H) 3.49-3.56 (m, 5 H) 3.83 (dd, J = 10.16, 3.64 Hz, 2 H) 3.90 (br. s., 4 H) 5.07 (dd, J = 10.10, 2.32 Hz, 2 H) 5.64-5.74 (m, 2 H) 5.78 (dd, J = 15.30, 11.30 Hz, 1 H) 6.12 (d, J = 10.79 Hz, 1 H) 6.35 (dd, J = 14.68, 10.54 Hz, 1 H) |

### N-Oxide Analogs

[chemical structure]

Compound 29 was prepared as follows:

Step S-30: A solution of I (E7107, 1.0 equiv.) in THF:H₂O (4:1, 0.01M) under nitrogen at 0° C. was added sodium metaperiodate (5.0 equiv.). The reaction was allowed to warm to room temperature and stirred for 7 days. The reaction was filtered and the filtrate was concentrated in vacuo and purified by HPLC (acetonitrile/0.1% aqueous formic acid as eluant) to afford the desired product (CC; Compound 29). ¹H NMR (400 MHz, METHANOL-d₄) ppm 0.87-0.94 (m, 6H) 0.96 (t, J=7.40 Hz, 3H) 1.18-1.32 (m, 5H) 1.34-1.42 (m, 5H) 1.45-1.71 (m, 12H) 1.75-1.91 (m, 8H) 233-2.44 (m, 2H) 2.50-2.56 (m, 2H) 2.56-2.64 (m, 1H) 2.66-2.71 (m, 2H) 2.87-2.94 (m, 1H) 3.00-3.10 (m, 2H) 3.35-3.41 (m, 6H) 3.48-3.57 (m, 1H) 3.60-3.73 (m, 2H) 3.73-3.84 (m, 1H) 4.97 (d, J=9.79 Hz, 1H) 5.08 (d, J=10.67 Hz, 1H) 5.61 (dd, J=15.18, 9.79 Hz, 1H) 5.75 (dd, J=15.31, 9.66 Hz, 1H) 5.89 (d, J=15.31 Hz, 1H) 6.16 (d, J=10.79 Hz, 1H) 6.55 (dd, J=15.25, 10.98 Hz, 1H)

[chemical structure]

[chemical structure]

[chemical structure]

Example 30 was prepared as follows:

Step S-31: A solution of (S)-3-bromo-2-methylpropan-1-ol DD (1.0 equiv.) in dichloromethane (0.3M) under nitrogen at 0° C. was added DMAP (0.20 equiv.), triethylamine (2.5 equiv.), and TBSOTf (1.2 equiv.). The reaction was allowed to warm to room temperature and stirred for 16 hours, or until the reaction was determined to be complete by LCMS or TLC. The reaction was washed with water and brine, dried over magnesium sulfate, filtered, and concentrated in vacuo. The resulting oil was purified by silica gel column chromatography (hexane/ethyl acetate as eluent) to afford the desired product (EE).

Step S-32: A solution of silyl ether EE (1.0 equiv.) in N,N-dimethylformamide (0.45M) under nitrogen at 0° C. was added NaH (1.0 equiv., 60% dispersion in mineral oil) and 1-phenyl-1H-tetrazole-5-thiol (1.0 equiv.). The reaction was allowed to warm to room temperature and then to 50° C. and stirred for 24 hours, or until the reaction was determined to be complete by LCMS or TLC. The reaction was cooled to 0° C. and quenched with water, washed with brine, dried over magnesium sulfate, filtered, and concentrated in vacuo. The resulting oil was purified by silica gel column chromatography (hexane/ethyl acetate as eluent) to afford the desired product (FF).

Step S-33: A solution of thioether FF (1.0 equiv.) in ethanol (0.11M) under nitrogen at 0° C. was added ammonium molybdate tetrahydrate (0.1 equiv.) and hydrogen peroxide (10.0 equiv., 30% solution in water). The reaction was allowed to warm to room temperature and stirred for 4 hours, or until the reaction was determined to be complete by LCMS or TLC. The reaction was diluted with ethyl acetate and washed with water and brine, dried over magnesium sulfate, filtered, and concentrated in vacuo. The resulting oil was purified by silica gel column chromatography (hexane/ethyl acetate as eluent) to afford the desired product (GG).

Step S-34: A solution of E7107 (I, 1.0 equiv.) under nitrogen in DMF (0.05M) at 0° C. was treated with imidazole (7.0 equiv.) and TBSCl (5.0 equiv.) was added. The reaction was allowed to warm to room temperature and stirred for 20 hours, or until the reaction was determined to be complete by LCMS or TLC. The reaction was extracted with ethyl acetate and the organic layer was washed with brine, dried over sodium sulfate, filtered, and concentrated in vacuo. The resulting oil was purified by silica gel column chromatography (hexanes/ethyl acetate as eluant) to afford the desired product (Q).

Step S-35: A solution of olefin HH (1.0 equiv.) in THF:H₂O (10:1, 0.01M) under nitrogen at 0° C. was added osmium tetroxide (0.2 equiv., 2.5% solution) followed by N-methylmorpholine N-oxide (2.0 equiv.). The reaction was allowed to warm to room temperature and stirred for 13 hours, or until the reaction was determined to be complete by LCMS or TLC. The reaction was quenched with sodium sulfite, diluted with ethyl acetate, and the organic layer was washed with water, dried, over magnesium sulfate, filtered, and concentrated in vacuo. The resulting oil was purified by silica gel column chromatography (dichloromethane/methanol as eluent) to afford the desired product (II).

Step S-36: A solution of diol II (1.0 equiv.) in acetone:H₂O (10:1, 0.03M) under nitrogen at room temperature was added (diacetoxyiodo)benzene (1.2 equiv.). The reaction was stirred for 30 minutes, or until the reaction was determined to be complete by LCMS or TLC. The reaction was quenched with sodium sulfite and diluted with dichloromethane. The organic layer was washed with water, dried over sodium sulfate, filtered, and concentrated in vacuo. The desired product (JJ) was advanced crude.

Step S-37: To a solution of sulfone GG (2.5 equiv.) in THF (0.02M) under nitrogen at −78° C. was added KHMDS (2.5 equiv.) dropwise and stirred for 10 minutes. Then aldehyde JJ (1.0 equiv.) in THF was added dropwise. The reaction was stirred at −78° C. for five hours and then allowed to warm to room temperature overnight. The reaction was diluted with ethyl acetate and washed with water and brine, dried over magnesium sulfate, filtered, and concentrated in vacuo. The resulting oil was purified by silica gel column chromatography (hexane/ethyl acetate as eluent) to afford the desired product (KK).

Step S-38: A solution of silyl ether KK (1.0 equiv.) in EtOH (0.01M) tinder nitrogen at room temperature was treated with PPTS (10.0 equiv.). The reaction was stirred for 16 hours, or until the reaction was determined to be complete by LCMS or TLC. The reaction was then diluted with ethyl acetate and washed with brine, dried over magnesium sulfate, filtered, and concentrated in vacuo. The resulting oil was purified by preparative TLC (dichloromethane/methanol as eluant) to afford the desired product (LL).

Step S-39: A solution of alcohol LL (1.0 equiv.) in dichloromethane (0.01M) under nitrogen at room temperature was treated with DMAP (1.0 equiv.), N,N-diisopropylethylamine (5.0 equiv.), and 4-nitrophenylchloroformate (2.0 equiv.). The reaction was stirred for 2 hours, or until the reaction was determined to be complete by LCMS or TLC. Next, N-methyl-N-propylamine (20.0 equiv.) was added at room temperature. The reaction was stirred at room temperature for 16 hours. The reaction was diluted with dichloromethane and washed with brine, dried over magnesium sulfate, filtered, and concentrated in vacuo. The resulting oil was purified by silica gel column chromatography (hexanes/ethyl acetate as eluant) to afford the desired product (MM).

Step. S-40: A solution of carbamate MM (1.0 equiv.) in methanol (0.02M) under nitrogen at room temperature was treated with PPTS (2.0 equiv.). The reaction was stirred for 1 hour, or until the reaction was determined to be complete by LCMS or TLC. The reaction was diluted with ethyl acetate and washed with brine, dried over magnesium sulfate, filtered, and concentrated in vacuo. The resulting oil was purified by preparative TLC (dichloromethane/methanol as eluant) to afford the desired product (NN; compound 30). ¹H NMR (400 MHz, METHANOL-d₄) δ ppm 0.79 (dd, J=7.03, 2.76 Hz, 14H) 0.97 (d, J=6.78 Hz, 4H) 1.05-1.34 (m, 30H) 1.65 (d, J=0.75 Hz, 10H) 1.91 (s, 1H) 2.08-2.32 (m, 5H) 2.43 (br. s., 5H) 2.84-3.01 (m, 3H) 3.07-3.15 (m, 3H) 3.25 (s, 2H) 3.43-3.63 (m, 4H) 3.63-3.77 (m, 1H) 3.81 (br. s., 3H) 4.37-4.53 (m, 2H) 4.95 (d, J=10.79 Hz, 1H) 5.39-5.70 (m, 3H) 5.89-6.08 (m, 1H) 6.25 (dd, J=14.56, 10.54 Hz, 1H)

[chemical structure]

[chemical structure]

[chemical structure]

Compound 31 was prepared as follows:

Note: Aldehyde OO was prepared as a product of Step S-36.

Step S-41: A solution of aldehyde OO (1.0 equiv.) in methanol (0.08M) under nitrogen at 0° C. was treated with Bestmann reagent (1.2 equiv.) over 15 minutes with a syringe pump. The reaction was stirred for 1 hour, or until the reaction was determined to be complete by LCMS or TLC. The reaction was diluted with diethyl ether and quenched with sodium bicarbonate, dried over magnesium sulfate, filtered, and concentrated in vacuo. The resulting oil was purified by silica gel column chromatography (hexanes/ethyl acetate as eluant) to afford the desired product (PP).

Step S-42: A solution of alkyne PP (1.0 equiv.) in DMF (0.1M) under nitrogen at room temperature was treated with imidazole (3.0 equiv.) followed by TESCl (2.0 equiv.). The reaction was stirred for 16 hours, or until the reaction was determined to be complete by LCMS or TLC. The reaction was quenched with sodium bicarbonate and diluted with ethyl acetate, and the organic layer was dried over magnesium sulfate, filtered, and concentrated in vacuo. The resulting oil was purified by silica gel column chromatography (hexanes/ethyl acetate as eluant) to afford the desired product (QQ).

Step S-43: A solution of alkyne QQ (1.0 equiv.) in 1,2-dichloroethane (0.1M) under nitrogen at room temperature was treated with pinacolborane (1.5 equiv.) and carbonylbis(triphenylphosphine)rhodium(I) chloride (0.2 equiv.). The reaction was heated to 40° C. and stirred for 12 hours, or until the reaction was determined to be complete by LCMS or TLC. The reaction was cooled to room temperature, quenched with water, and diluted with ethyl acetate. The organic layer was dried over magnesium sulfate, filtered, and concentrated in vacuo. The resulting oil was purified by silica gel column chromatography (hexanes/ethyl acetate as eluant) to afford the desired product (RR).

Note: Vinyl iodide SS was prepared as shown in Burkart, M. D. et al. Bioorg. Med. Chem. Lett. 2007, 17, 5159-5164.

Step S-44: A solution of macrocycle SS (1.0 equiv.) in dioxane (0.02M) under nitrogen at room temperature was treated with selenium dioxide (2.0 equiv.). The reaction was heated to 80° C. and stirred for 1.5 hours, or until the reaction was determined to be complete by LCMS or TLC. The reaction was diluted with ethyl acetate, and the organic layer was washed with sodium bicarbonate solution, dried over magnesium sulfate, filtered, and concentrated in vacuo. The resulting oil was purified by silica gel column chromatography (hexanes/ethyl acetate as eluant) to afford the desired product (TT).

Step S-45: A solution of alcohol TT (1.0 equiv.) in methyl tert-butyl ether (0.08M) under nitrogen at room temperature was treated with DMAP (1.2 equiv.), triethylamine (5.0 equiv.), and 4-nitrophenylchloroformate (3.0 equiv.). The reaction was stirred for 16 hours, or until the reaction was determined to be complete by LCMS or TLC. The reaction was diluted with ethyl acetate and washed with brine, dried over magnesium sulfate, filtered, and concentrated in vacuo. The resulting oil was purified by silica gel column chromatography (hexanes/ethyl acetate as eluant) to afford the desired product (UU).

Step S-46: A solution of carbonate UU (1.0 equiv.) in methyl tert-butyl ether (0.01M) under nitrogen at room temperature was treated with 1-cycloheptylpiperazine (1.5 equiv.). The reaction was stirred for 16 hours, or until the reaction was determined to be complete by LCMS or TLC. The reaction was diluted with ethyl acetate and washed with brine, dried over magnesium sulfate, filtered, and concentrated in vacuo. The resulting oil was purified by silica gel column chromatography (hexanes/ethyl acetate as eluant) to afford the desired product (VV).

Step S-47: A solution of iodide VV (1.0 equiv.) in THF (0.09M) under nitrogen at room temperature was treated with silver (I) oxide (5.0 equiv.), triphenylarsine (1.0 equiv.), tris(dibenzylideneacetone)dipalladium(0) (0.2 equiv.), and pinacol boronate (RR). The reaction was stirred for 16 hours, or until the reaction was determined to be complete by LCMS or TLC. The reaction was filtered through Celite®, washed with dichloromethane, and concentrated. The resulting oil was purified by silica gel column chromatography (hexanes/ethyl acetate as eluant) to afford the desired product (WW).

Step S-48: A solution of silyl ether WW (1.0 equiv.) in DMF (0.01M) under nitrogen at room temperature was treated with TBAF (5.0 equiv.) and water (5.0 equiv.). The reaction was stirred for 16 hours, or until the reaction was determined to be complete by LCMS or TLC. The reaction was diluted with ethyl acetate and the organic layer was dried over magnesium sulfate, filtered, and concentrated in vacuo. The resulting oil was purified by silica gel column chromatography (dichloromethane/methanol as eluant) to afford the desired product (XX).

Step S-49: A solution of carbamate XX (1.0 equiv.) in dichloromethane (0.01M) under nitrogen at room temperature was treated with methyl iodide (100 equiv.). The reaction was stirred for 60 hours, or until the reaction was determined to be complete by LCMS or TLC. The reaction was concentrated in vacuo and purified by silica gel column chromatography (dichloromethane/methanol as eluant) to afford the desired product (YY; compound 31). ¹H NMR (400 MHz, METHANOL-d₄) δ ppm 0.77 (d, J=6.78 Hz, 3H) 0.80 (d, J=7.28 Hz, 3H) 0.84 (t, J=7.28 Hz, 3H) 1.10-1.22 (m, 8H) 1.25 (s, 3H) 1.34-1.51 (m, 6H) 1.53-1.60 (m, 2H) 1.68 (s, 3H) 1.70-1.80 (m, 8H) 2.05-2.18 (m, 3H) 2.22-234 (m, 1H) 2.43-2.53 (m, 1H) 2.55-2.60 (m, 1H) 2.75-183 (m, 1H) 2.90 (s, 3H) 3.31-3.68 (m, 8H) 3.76-4.01 (m, 1H) 4.94 (d, J=10.54 Hz, 1H) 4.97-5.06 (m, 1H) 5.21-5.50 (m, 2H) 5.78 (d, J=15.31 Hz, 1H) 6.03 (d, J=11.29 Hz, 1H) 6.27-6.60 (m, 1H)

### Biological Activity

### Examples 32-62

### Cell Viability Assay Protocol

Cells are seeded in 96-well plate with 2000 cells/100 uL/well and incubated overnight. Spent media was removed and fresh media containing compound (100 uL/well) with appropriate 9 concentrations are added. Each compound was treated in duplicate or triplicate manner at each concentration. DMSO concentration was adjusted to be 0.1%. Another plate was dedicated as Tz plate which was added 0.1% DMSO in media (100 uL/well) followed by CellTiter-Glo® (Promega corp., 50 uL/well) for ATP measurement as a surrogate of cell viability. Values from this plate are used as Tz. Compound treated plates are incubated for 72 hr at 37 C. Then, CellTiter-Glo® (50 uL/well) was added and ATP level was measured. Values from compound treated wells are used as Ti and DMSO treated wells are used as C.

Percentage growth inhibition was calculated as:

[(Ti−Tz)/(C−Tz)]×100 for concentrations for which Ti>/=Tz

[(Ti−Tz)/Tz]×100 for concentrations for which Ti<Tz.

*[time zero, (Tz), control growth (C), and test growth in the presence of drug with dose (Ti)] Growth inhibition of 50% (GI50) was calculated from [(Ti−Tz)/(C−Tz)]×100=50, which was the drug concentration resulting in a 50% reduction in the net protein increase in control cells during the drug incubation. The LC50 (concentration of drug resulting in a 50% reduction in the measured value at the end of the drug treatment as compared to that at the beginning) indicating a net loss of cells following treatment was calculated from [(Ti−Tz)/Tz]×100=−50.

Eₘₐₓ was defined as highest growth inhibition that particular compound achieved in tested dose range.

In Vitro Splicing (Biochemical) Assay Protocol

Biotin-labeled pre-mRNA of an adenovirus type 2 construct with a deletion of intervening sequence (Ad2)¹ was prepared by in vitro transcription. The Ad2 construct containing Exon 1 (41 nt), Intron (231 nt), and Exon 2 (72 nt) was generated by gene synthesis and cloned into the EcoRI and XbaI sites of pGEM-3Z (Promega) by Genewiz. The plasmid was then linearized by XbaI digestion and purified. In vitro transcription and purification of transcribed pre-mRNA were performed using the MEGAScript T7 kit (Invitrogen) and MEGAclear kit (Invitrogen), respectively, following the manufacturer instructions. The ratio of biotin-16-UTP (Roche) to cold UTP was 1:13 to incorporate approximately two biotin molecules per spliced Ad2 mRNA.

In vitro splicing assay was performed at 30° C. in 25 μL reaction mixtures containing 95 μg HeLa nuclear extract (Promega), 47 nM Ad2 pre-mRNA, 25 U RNasin RNase inhibitor (Promega), 1×SP buffer (0.5 mM ATP, 20 mM creatine phosphate, 1.6 mM MgCl₂), and compounds in DMSO (with 1% final concentration of DMSO). After 90 min of incubation, the reaction was stopped by addition of 18 μL of 5M NaCl, and the mixtures were incubated with 10 μL of M-280 streptavidin-coated magnetic beads (Invitrogen) for 30 min at room temperature to capture Ad2 pre- and spliced mRNA. The beads were washed twice with 100 uL buffer containing 10 mM Tris pH=7.5, 1 mM EDTA and 2M NaCl, and then incubated in RNA gel loading buffer containing 95% formamide at 70° C. for 10 min to elute the RNAs. Ad2 RNAs were resolved by 6% TBE-UREA gel, transferred to a nylon membrane, UV cross-linked, and probed with an IR-dye labeled streptavidin (LiCor) using LiCor detection. The amount of spliced RNA was quantified by measuring the band fluorescent intensity using LiCor software.

### REFERENCES

1. Berg, M. G., et al. 2012 Mol. Cell. Bio., 32(7):1271-83

TABLE 5
Examples 32-62

| | | | | Gl₅₀ | | Bio | |
|---|---|---|---|---|---|---|---|
| | Example | Gl₅₀ | Gl₅₀ | Panc | Eₘₐₓ | chem | |
| | (Compound | WiDr | WiDr-R | 05.04 | Panc | IC₅₀ | |
| Compound | No.) | (nM) | (nM) | (nM) | 05.04 | (nM) | ES⁺ |
| [chemical structure] | 32 (Cpd. 17) | 64.5 | >1000 | 607.8 | −47% | <100 | 733.8 |
| [chemical structure] | 33 (cpd. 18) | 162 | | 139.2 | −55% | | 809.7 |
| [chemical structure] | 34 (Cpd. 19) | 551 | | 808.7 | −45% | | 759.7 |
| [chemical structure] | 35 (Cpd. 20) | 289 | | 316.9 | −67.5 | | 759.7 |
| [chemical structure] | 36 (Cpd. 24) | 31.3 | | 41.6 | −49% | <40 | 829.7 |
| [chemical structure] | 37 (Cpd. 23) | >10000 | >10000 | 10000 | | | 747.7 |
| [chemical structure] | 38 (Cpd. 1) | 1.1 | 368 | 0.6 | −61% | 10 | 717.4 |
| [chemical structure] | 39 (Cpd. 2) | 8.9 | >1000 | 17.0 | −69% | 50 | 635.4 |
| [chemical structure] | 40 (Cpd. 3) | 2.8 | >100 | 16.2 | −66% | 60 | 663.6 |
| [chemical structure] | 41 (Cpd. 4) | 67.4 | | 41.5 | −75% | | 679.5 |
| [chemical structure] | 42 (Cpd. 5) | 176.0 | >10000 | 120.8 | −79% | | 637.4 |
| [chemical structure] | 43 (Cpd. 21) | 663.0 | >10000 | 857.9 | −38% | | 731.7 |
| [chemical structure] | 44 (Cpd. 22) | 350.0 | >10000 | 599.2 | −47% | | 745.6 |
| [chemical structure] | 45 (Cpd. 26) | 1491.0 | >10000 | 5305 | | | 759.5 |
| [chemical structure] | 46 (Cpd. 6) | 227.0 | >1000 | 1593 | −8.1 | | 703.7 |
| [chemical structure] | 47 (Cpd. 7) | 7.5 | >10000 | 7.0 | −64% | | — |
| [chemical structure] | 48 (Cpd. 27) | 6781.0 | >10000 | 1115 | −56% | | 705.6 |
| [chemical structure] | 49 (Cpd. 25) | 5500.0 | >5500 | 10000 | | | 731.7 |
| [chemical structure] | 50 (Cpd. 8) | 568.0 | >1000 | 431.6 | −74% | | 649.5 |
| [chemical structure] | 51 (Cpd. 9) | >1000 | >1000 | 10000 | | | — |
| [chemical structure] | 52 (Cpd. 10) | 1.9 | >1000 | 1.3 | −65% | 60 | 677.6 |
| [chemical structure] | 53 (Cpd. 11) | 958.0 | >1000 | 410.7 | −87% | | 717.6 |
| [chemical structure] | 54 (Cpd. 12) | 106.0 | >1000 | 79.2 | −89% | | 729.5 |
| [chemical structure] | 55 (Cpd. 28) | 456.0 | >1000 | 413.3 | −31% | | — |
| [chemical structure] | 56 (Cpd. 13) | 44.1 | >1000 | 80.4 | −61% | | 689.7 |
| [chemical structure] | 57 (Cpd. 14) | 51.4 | >1000 | 53.3 | −70% | | 703.7 |
| [chemical structure] | 58 (Cpd. 15) | 117.0 | >1000 | 234.8 | −53% | | 663.7 |
| [chemical structure] | 59 (Cpd. 16) | >1000 | >1000 | 1746 | −27% | | 647.3 |
| [chemical structure] | 60 (Cpd. 29) | 445 | >10000 | 113.7 | −10% | | 734.5 |
| [chemical structure] | 61 (Cpd. 30) | 172 | >10000 | 76.1 | −86% | 10000 | 706.6 |
| [chemical structure] | 62 (Cpd. 31) | 3285 | >10000 | 2458 | −22% | | 687.4 |

Key
WiDr cells: Colon cancer cells (wildtype SF3B1)
WiDr-R cells: Colon cancer cells (chemically-induced SF3B1 mutant which is resistant to E7107 (R1074H mutation))
Panc 05.04 cells: Pancreatic cancer cells (Q699H and K700E mutations in SF3B1)
LCMS information
Mobile phases: A (0.1% formic acid in H₂O) and B (0.1% folic acid in acetonitrile)
Gradient: B 5% → 95% in 1.8 minutes
Column: Acquity BEH C18 column (1.7 um, 2.1 × 50 mm)

Additional compounds made and tested included the following:

TABLE 6
COMPOUND

[chemical structure]

[chemical structure]

[chemical structure]

Biological activity is confirmed in additional cell lines:

AsPC1 (pancreatic cancer, wt SF3B1);

ESS-1 (endometrial cancer, K666N mt SF3B1);

AN3CA (endometrial cancer, wt SF3B1);

Nalm-6 (pre-B-cell leukemia isogenic cell line with wt SF3B1);

Nalm-6 (pre-B-cell leukemia isogenic cell line with K700E mt SF3B1).

### Other Embodiments

While we have described a number of embodiments of this invention, these basic examples may be altered to provide other embodiments that utilize the compounds and methods of this invention.

1. A quaternary salt of Formula I: [chemical structure]
wherein:
Y is a quaternary nitrogen;
X⁻ is a pharmaceutically acceptable anion;
L¹ is alkylene or alkenylene; or L¹ and R¹² are joined together to form a branched alkylene or alkenylene;
L² is H or alkyl; or L² and R¹² are joined together to form an alkylene or alkenylene; or
L² and L¹ are joined together to form a branched alkylene or alkenylene;
or L¹, L² and R¹² are joined together, with the two nitrogens to which they are attached, to form a spiro heterocycle or a bridged heterocycle;
R¹² is alkyl, or R¹² is joined to L¹ and/or L²;
R¹⁰ and R¹¹ are each independently: alkyl, alkenyl, cycloalkyl, aryl, arylalkyl, arylalkenyl, and heterocycle; or R¹⁰ and R¹¹ are taken together with the nitrogen to which they are attached to form a heterocycle;
R¹ is hydrogen, hydroxy, alkoxy, C₁₋₆ alkyl ester, or alkyl;
R² is H or alkyl;
R³ is H, alkyl, or hydroxy;
R⁴, R⁵ and R⁸ are each independently C₁-C₆ alkyl;
R⁶ and R⁷ are each independently selected from the group consisting of H, hydroxy, and alkyl such as methyl; and
R⁹ is hydroxy or oxo;
or a pharmaceutically acceptable prodrug thereof,
wherein said alkylene, alkenylene, alkyl, alkenyl, cycloalkyl, aryl, arylalkyl, arylalkenyl, and heterocycle groups may each independently be unsubstituted or substituted 1-3 times.

2. The quaternary salt of claim 1, wherein said salt is a salt of Formula I(a): [chemical structure]
wherein:
X⁻ is a pharmaceutically acceptable anion;
R¹, R², R³, R⁴, R⁵, R⁶, R⁷, R⁸, and R⁹ are as defined above;
L¹ and L² are each independently C₁-C₆ alkylene or C₂-C₆ alkenylene; or L¹ and L² are taken together with the two nitrogens to which they are attached to form a bridged heterocyclic moiety;
R¹⁰ and R¹¹ are each independently selected from the group consisting of: C₁-C₆ alkyl, C₂-C₆ alkenyl, C₃-C₈ cycloalkyl, and C₃-C₈ heterocycle; or R₁₀ and R₁₁ are taken together with the nitrogen to which they are attached to form a 5-7 membered heterocycle;
or a pharmaceutically acceptable prodrug thereof.

3. The quaternary salt of claim 1, wherein said salt is a salt of Formula I(b): [chemical structure]
wherein:
X⁻ is a pharmaceutically acceptable anion;
R¹, R², R³, R⁴, R⁵, R⁶, R⁷, R⁸, and R⁹ are as defined above;
L¹ is C₁-C₆ alkylene or C₂-C₆ alkenylene;
R¹³ is selected from the group consisting of: hydrogen, C₁-C₆ alkyl, C₂-C₆ alkenyl, C₃-C₈ cycloalkyl, and C₃-C₈ heterocycle;
R¹⁰ is selected from the group consisting of: C₁-C₆ alkyl, C₂-C₆ alkenyl, C₃-C₈ cycloalkyl, and C₃-C₈ heterocycle;
R¹¹ and R¹² are each independently selected from the group consisting of: C₁-C₆ alkyl, C₂-C₆ alkenyl, C₃-C₈ cycloalkyl and C₃-C₈ heterocycle; or R₁₁ and R₁₂ are taken together with the nitrogen to which they are attached to form a 4-7 membered heterocycle,
or a pharmaceutically acceptable prodrug thereof.

4. The quaternary salt of claim 1, wherein said salt is a salt of Formula I(c): [chemical structure]
wherein:
X⁻ is a pharmaceutically acceptable anion;
R¹, R², R³, R⁴, R⁵, R⁶, R⁷, R⁸, and R⁹ are as defined above;
L¹ and L² are each independently C₁-C₆ alkylene or C₂-C₆ alkenylene; or L¹ and L² are taken together with the nitrogen to which they are attached to form a bridged heterocyclic moiety;
L⁴ is a direct bond, a C₁-C₆ alkylene, or a C₂-C₆ alkenylene;
R¹² is selected from the group consisting of hydrogen, C₁-C₆ alkyl, C₂-C₆ alkenyl, C₃-C₈ cycloalkyl, and C₃-C₈ heterocycle;
R¹³ is selected from the group consisting of C₁-C₆ alkyl, C₂-C₆ alkenyl, C₃-C₈ cycloalkyl, and C₃-C₈ heterocycle;
R¹⁴ and R¹⁵ are each independently selected from the group consisting of C₁-C₆ alkyl, C₂-C₆ alkenyl, C₃-C₈ cycloalkyl, and C₃-C₈ heterocycle; or R¹⁴ and R¹⁵ are taken together with the nitrogen to which they are attached to form a 4-7 membered heterocycle,
or a pharmaceutically acceptable prodrug thereof.

5. The quaternary salt of claim 1, wherein said salt is a salt of Formula I(d): [chemical structure]
wherein:
X⁻ is a pharmaceutically acceptable anion;
R¹, R², R³, R⁴, R⁵, R⁶, R⁷, R⁸, and R⁹ are as defined above;
L⁵, L⁶, L⁷, and L⁸ are each independently C₁-C₄ alkylene;
R¹⁰ is selected from the group consisting of: C₁-C₆ alkyl, C₂-C₆ alkenyl, C₃-C₈ cycloalkyl, and C₃-C₈ heterocycle;
R¹¹ is selected from the group consisting of C₁-C₆ alkyl, C₂-C₆ alkenyl, C₃-C₈ cycloalkyl, and C₃-C₈ heterocycle, wherein said alkyl, alkenyl, or cycloalkyl groups may be unsubstituted or substituted with an aryl,
or a pharmaceutically acceptable prodrug thereof.

6. The quaternary salt of claim 1, wherein said salt is a salt of Formula I(e): [chemical structure]
wherein:
X⁻ is a pharmaceutically acceptable anion;
R¹, R², R³, R⁴, R⁵, R⁶, R⁷, R⁸, and R⁹ are as defined above;
L¹ and L² are each independently C₁-C₆ alkylene or alkenylene;
R¹² and R¹⁶ are each independently selected from the group consisting of: hydrogen, C₁-C₆ alkyl, C₂-C₆ alkenyl, C₃-C₈ cycloalkyl, and C₃-C₈ heterocycle;
R¹⁰ is selected from the group consisting of: C₁-C₆ alkyl, C₂-C₆ alkenyl, C₃-C₈ cycloalkyl, and C₃-C₈ heterocycle; and
R¹¹ is selected from the group consisting of C₁-C₆ alkyl, C₂-C₆ alkenyl, C₃-C₈ cycloalkyl, and C₃-C₈ heterocycle, wherein said alkyl, alkenyl, or cycloalkyl groups may be unsubstituted or substituted with an aryl,
or a pharmaceutically acceptable prodrug thereof.

7. The quaternary salt of claim 1, wherein said salt is selected from the group consisting of: [chemical structure] [chemical structure] [chemical structure] [chemical structure] [chemical structure]
wherein X⁻ is a pharmaceutically acceptable anion;
or pharmaceutically acceptable prodrug thereof.

8. The salt of claim 1, wherein X⁻ is Br⁻.

9. The salt of claim 1, wherein X⁻ is I⁻.

10. The quaternary salt of claim 1, wherein said salt is selected from the group consisting of: [chemical structure]
wherein X⁻ is a pharmaceutically acceptable anion;
or pharmaceutically acceptable prodrug thereof.

11. The salt of claim 10, wherein X⁻ is Br⁻.

12. The salt of claim 10, wherein X⁻ is I⁻.

13. An amine oxide of Formula II: [chemical structure]
wherein:
L¹ and L² are each independently C₁-C₆ alkylene or C₂-C₆ alkenylene;
one of either R¹⁰ or R¹¹ is oxygen, and the other is selected from the group consisting of: C₁-C₆ alkyl, C₂-C₆ alkenyl, C₃-C₈ cycloalkyl, and C₃-C₈ heterocycle;
R¹ is hydrogen, hydroxy, C₁-C₆ alkoxy, C₁₋₆ alkyl ester, or C₁-C₆ alkyl;
R² is H or C₁-C₆ alkyl;
R³ is H, C₁-C₆ alkyl, or hydroxy;
R⁴, R⁵ and R⁸ are each independently C₁-C₆ alkyl;
R⁶ and R⁷ are each independently selected from the group consisting of: H, hydroxy, and C₁-C₆ alkyl; and
R⁹ is hydroxy or oxo;
or a pharmaceutically acceptable prodrug thereof.

14. The amine oxide of claim 13, wherein said amine oxide is: [chemical structure]
or a pharmaceutically acceptable prodrug thereof.

15. A quaternary ammonium salt of Formula III: [chemical structure]
wherein:
X⁻ is a pharmaceutically acceptable anion;
L¹ and L² are each independently C₁-C₆ alkylene or C₂-C₆ alkenylene; or L¹ and L² are taken together with the two nitrogens to which they are attached to form a bridged heterocycle;
R¹⁰ is selected from the group consisting of: C₁-C₆ alkyl, C₂-C₆ alkenyl, C₃-C₈ cycloalkyl, and C₃-C₈ heterocycle;
R¹¹ is selected from the group consisting of: C₁-C₆ alkyl, C₂-C₆ alkenyl, C₃-C₈ cycloalkyl, and C₃-C₈ heterocyclyl; wherein said alkyl, alkenyl, or cycloalkyl groups may be unsubstituted or substituted with an aryl;
R² is H or C₁-C₆ alkyl;
R³ is H, C₁-C₆ alkyl, or hydroxy;
R⁴, R⁵ and R⁸ are each independently C₁-C₆ alkyl;
R⁶, R⁷ and R⁹ are each independently selected from the group consisting of H, hydroxy, and C₁-C₆ alkyl;
or a pharmaceutically acceptable prodrug thereof.

16. The quaternary salt of claim 13, wherein said salt is selected from the group consisting of: [chemical structure]
wherein X⁻ is a pharmaceutically acceptable anion;
or pharmaceutically acceptable prodrug thereof.

17. The salt of claim 15, wherein X⁻ is Br⁻.

18. The salt of claim 15, wherein X⁻ is I⁻.

19. An amine oxide of Formula IV: [chemical structure]
wherein:
L¹ and L² are each independently C₁-C₆ alkylene or C₂-C₆ alkenylene; or L¹ and L² are taken together with the two nitrogens to which they are attached to form a bridged heterocyclic moiety;
one of either R¹⁰ or R¹¹ is oxygen, and the other is selected from the group consisting of: C₁-C₆ alkyl, C₂-C₆ alkenyl, C₃-C₈ cycloalkyl, and C₃-C₈ heterocycle;
R¹ is hydrogen, hydroxy, alkoxy, C₁₋₆ alkyl ester, or C₁-C₆ alkyl;
R² and R⁴ are each independently H or C₁-C₆ alkyl;
R³ is H, C₁-C₆ alkyl, or hydroxy;
R⁵ is a C₁-C₆ alkyl;
R⁶ and R⁷ are each independently selected from the group consisting of H, hydroxy, and C₁-C₆ alkyl;
Y is O or N(R¹⁷)(R¹⁸);
R¹⁷ is selected from the group consisting of hydrogen, C₁-C₆ alkyl, C₂-C₆ alkenyl, C₃-C₈ cycloalkyl, C₃-C₈ heterocycle, and —(C═O)—N(R¹⁸)(R¹⁹);
R¹⁸ and R¹⁹ are each independently selected from the group consisting of hydrogen, C₁-C₆ alkyl, C₂-C₆ alkenyl, C₃-C₈ cycloalkyl, and C₃-C₈ heterocycle,
or a pharmaceutically acceptable prodrug thereof.

20. The amine oxide of claim 19, wherein said amine oxide is: [chemical structure]
or pharmaceutically acceptable prodrug thereof.

21. A pharmaceutical composition comprising the salt or amine oxide of claim 1; and a pharmaceutically acceptable carrier.

22. The pharmaceutical composition of claim 21, wherein said composition is formulated for intravenous, oral, subcutaneous, or intramuscular administration.

23. A method of treating cancer in a subject in need thereof, comprising administering to said subject an effective amount of a salt or amine oxide of claim 1, or a pharmaceutically acceptable prodrug thereof.

24. The method of claim 23, wherein the cancer is selected from the group consisting of myelodysplastic syndrome, chronic lymphocytic leukemia, chronic myelomonocytic leukemia, acute myeloid leukemia, colon cancer, pancreatic cancer, endometrial cancer, ovarian cancer, and breast cancer.

25. The method of claim 23, wherein the cancer is colon cancer or pancreatic cancer.

26. The method of claim 23, wherein said subject has cancer that is positive for mutations in the Splicing factor 3B subunit 1 (SF3B1) protein.

27-36. (canceled)
